(19) United States
(12) Patent Application Publication
(10) Pub. No.: US 2007/0264208 A1
(43) Pub. Date: Nov. 15, 2007

(54) Cosmetic and/or pharmaceutical composition comprising at least one copolymer comprising at least one ionizable group, and cosmetic treatment process

(76) Inventors: Nathalie Mougin, Paris (FR); Sandrine Chodorowski-Kimmes, Senlis (FR); Xavier Schultze, Pontault-Combault (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/640,420

(22) Filed: Dec. 18, 2006

Related U.S. Application Data
(60) Provisional application No. 60/754,183, filed on Dec. 28, 2005.

(30) Foreign Application Priority Data
Dec. 16, 2005 (FR) 05 53909

(51) Int. Cl. A61K 31/74; A61K 31/745; A61K 31/765; A61K 31/787; A61K 8/84; A61K 8/90

(52) U.S. Cl. 424/59; 424/70.2; 424/78.08; 424/78.1; 424/78.31; 424/78.36; 424/78.37

(57) ABSTRACT

Disclosed herein is a cosmetic and/or pharmaceutical composition comprising, in a physiologically acceptable medium, at least one (co)polymer comprising a polymer backbone and at least one junction group linked to the polymer backbone and capable of establishing H bonds with at least one partner junction group, each pairing of a junction group involving at least 3 H bonds, wherein the at least one (co)polymer comprises at least one ionizable group. Also disclosed herein is a cosmetic treatment process comprising applying the composition to keratin materials.

This application claims benefit of U.S. Provisional Application No. 60/754,183, filed Dec. 28, 2005, the contents of which are incorporated herein by reference. This application also claims benefit of priority under 35 U.S.C. § 119 to French Patent Application No. FR 05 53909, filed Dec. 16, 2005, the contents of which are also incorporated herein by reference.

Disclosed herein is a cosmetic composition for caring for, treating, and/or making up keratin materials, which may display increased persistence of at least one cosmetic and/or care effect provided by the composition after application, good adhesion after application to keratin materials, and may allow for rapid, total, and/or selective makeup removal.

In at least one embodiment, this composition may be film-forming and after application may lead to the formation of a film on the composition on drying.

In cosmetics, it is commonly desirable to obtain a deposit on the hair, the skin, the eyelashes, and/or the nails, which may be film-forming and which may provide, for example, shaping of the hair style (hair); color (hair and/or makeup); gloss and/or color (lipstick, mascaras, eyeliners, and/or nail varnish); color and mattness (for example, foundation); care effects, if the deposit contains an active care agent such as a moisturizer, and/or protection against UV, if it contains sunscreens.

Thus, for persistence of the provided effect (color, gloss, mattness, hold, and/or care effect), greater remanence is sought for the cosmetic deposit, which should withstand, for example, mechanical attacks such as rubbing, transfers by contact with another object; be resistant to water, sweat, tears, and/or rain; and/or be resistant to sebum and/or oils.

Persistence of the provided effect is often sought in makeup for lipsticks, where prolonged staying power of the color and gloss, and transfer resistance of the color are sought; nail varnishes, where gloss and staying power of the film are sought; and foundations, eyeshadows, and powders, where staying power of the provided color is sought, while maintaining the mattness of the initial shade for as long as possible despite the secretion of sebum and sweat, and also transfer resistance.

In the case of makeup compositions, it would be desirable to have available polymers of different or identical chemical nature, which are readily soluble or dispersible in oils, cosmetic solvents, and/or aqueous media, at variable concentration. It would also be desirable for these compositions to have increased persistence of the provided effects (for instance, color, gloss, mattness, and care effects), and for them to promote adhesion to keratin materials.

In styling, the composition ideally should allow shaping of the hair with good hold over time and under the temperature and humidity conditions of daily life.

To solve these problems, it has been proposed in the prior art to use in cosmetic compositions polymers capable of inducing, at least among themselves, physical crosslinking, for example, polymers comprising groups capable of forming hydrogen bonds.

Thus, International Patent Application Publication No. WO 02/098 377 describes a cosmetic composition for caring for, treating, or making up keratin materials comprising, in a physiologically acceptable medium, a linear, branched, cyclic, or dendrimetric polymer comprising a polymer backbone and a junction group capable of forming hydrogen bonds. The polymer backbones may comprise poly(ethylene oxide) (PEO) units.

Also, International Patent Application Publication No. WO 03/032 929 describes a hair treatment composition containing a polymer comprising at least two groups capable of forming hydrogen bonds.

U.S. Pat. No. 5,919,441 describes cosmetic compositions comprising a silicone oil and, as gelling agent, a polymer comprising siloxane groups and groups containing hydrogen bonds.

Conveying polymers in aqueous media, especially in water or in a medium comprising water, while at the same time maintaining the targeted properties of the compositions, for instance, those induced by the said added polymers, may prove to be difficult. For example, in the field of styling, problems may arise because a sufficient provision of styling effect by the polymers conveyed by the aqueous medium is necessary, while at the same time it is important to maintain good resistance over time of the effect.

Conveying polymers in non-aqueous media, such as organic solvents and cosmetic oils, may also prove to be difficult, for instance, in the field of makeup products such as foundations and lipsticks, and also nail varnishes.

As used herein, the expression “conveying a polymer in an aqueous or non-aqueous medium” means that the said polymer is soluble and/or dispersible in the said medium, at 25° C., at a concentration of at least 1% by weight.

There is a need for cosmetic compositions that give, after application to keratin materials, deposits that can exhibit at least one of persistence of the cosmetic and/or care effect, good adhesion of the composition to keratin materials, and total makeup removal, and that comprise polymers which are easy to convey in the physiologically acceptable medium of the composition.

Thus disclosed herein is a cosmetic composition, and in at least one embodiment, a cosmetic composition for caring for, cosmetically treating, and/or making up keratin materials, which may make it possible to overcome at least one of the drawbacks of conventional formulations.

The present inventors have found that a cosmetic composition comprising at least one polymer comprising a polymer backbone comprising at least two repeating units, at least one junction group bonded to the polymer backbone, this junction group being capable of interacting with any partner junction group via the formation of at least three H bonds (hydrogen bonds), shows improved staying power on a keratin support, for instance, on the nails.

Moreover, this composition may make it possible to obtain persistence of at least one cosmetic and/or care effect provided to keratin materials by the composition after application, or even to increase this effect.

Disclosed herein, therefore, is a cosmetic composition comprising, in a physiologically acceptable medium, at least one (co)polymer comprising:

(a) a polymer backbone -POL-, and

(b) at least one junction group (A) bonded to the polymer backbone and capable of establishing H bonds with at least one partner junction group, of identical or different chemical nature, each pairing of a junction group involving at least three H bonds, and in at least one embodiment, at least 4 H bonds, for example 4 H bonds, wherein the at least one (co)polymer comprises at least one ionizable group.

It has been found that the (co)polymers according to the present disclosure may be readily soluble and/or dispersible in the medium of the composition, for instance, the aqueous and/or oily and/or organic solvent media conventionally used in cosmetics.

Moreover, the polymer according to the present disclosure may allow for the production of a cosmetic composition whose removal is facilitated, when compared with compositions of the prior art, for example, those described in International Patent Application Publication No. WO 02/98377. This may be useful in the case of hair compositions, which must be easy to remove on shampooing.

In addition, the compositions according to the present disclosure may not have a tacky nature.

Finally, it has been found that compositions comprising polymers according to the present disclosure may have a low viscosity, although comprising large amounts of polymers; for example, compositions in an aqueous medium may exhibit a low viscosity. These compositions may also conserve good sprayability and may allow the production of a film-forming deposit on the support.

As used herein, the term “polymer backbone”, also referred to as POL, means a homopolymer or copolymer, denoted below as “(co)polymer”, comprising at least two identical or different covalently bonded repeating units. The (co)polymer may be chosen from linear, cyclic, branched, for instance, in star, dendrimer, or graftedform, and crosslinked polymers; it may be a homopolymer or a copolymer chosen from statistical copolymers, alternating copolymers, block copolymers, and the like.

As used herein, the term “junction group”, also referred to as A, means any functional group comprising H bond donor or acceptor groups, and capable of establishing at least three H bonds, and in at least one embodiment, at least 4 H bonds, for example, 4 H bonds, with an identical or different partner junction group.

The junction groups (A) may be lateral to the polymer backbone (as lateral branching) and/or at the ends of the said polymer backbone, and/or in the chain forming the said polymer backbone.

They may be distributed in a random or controlled manner in the chain.

As used herein, the term “partner junction group” means any junction group of a (co)polymer according to the present disclosure that can establish H bonds with at least one junction group of the same or of another (co)polymer according to the present disclosure. The junction groups may be of identical or different chemical nature. If they are identical, they can then establish H bonds between themselves and are then referred to as self-complementary junction groups. If they are different, they are chosen such that they are complementary with respect to H interactions.

The use of such (co)polymers in a cosmetic composition leads, after application of this composition to keratin materials, to the formation of a supramolecular polymer.

As used herein, the term “supramolecular polymer” means a polymer chain or network formed from the assembly of a (co)polymer according to the present disclosure with at least one other identical or different (co)polymer according to the present disclosure, each assembly comprising at least one pair of identical or different paired junction groups.

As used herein, the term “pair of paired junction groups” means two junction groups, each of which may or may not be borne by the same (co)polymer according to the present disclosure, the two groups being bonded together via at least three H bonds, and in at least one embodiment, at least 4 H bonds, for example, 4 H bonds.

Thus, the supramolecular polymer will have physical crosslinking points provided by the H bonds between these pairs of junction groups. The physical crosslinking will ensure the maintenance and persistence of the cosmetic and/or care effect analogously with chemical crosslinking, while at the same time allowing reversibility, i.e. the possibility of totally removing the deposit, for instance, by specific makeup removal, by temperature, and/or by any other means, which chemical crosslinking does not allow.

As used herein, the term “ionizable group” means any group which, either by its intrinsic chemical nature, or as a function of the medium and/or of the pH of the medium in which it is present, may be in ionic form. Depending on its chemical nature, it may be chosen from cationizable, anionizable, and amphoteric groups. This also includes, for example, tetra-N-substituted quaternary ammonium ionic groups.

The at least one (co)polymer according to the present disclosure may be defined as resulting from the homopolymerization or copolymerization of monomers of formula (I) and optionally of monomers of formula (II) according to the following reaction scheme: 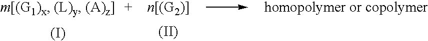
wherein:

the groups G₁, which may be identical or different, are (co)polymerizable groups capable of forming a covalent bond with another (co)polymerizable group G₁ of another monomer (I) and/or with a (co)polymerizable group borne by a monomer G₂;

the groups A, which may be identical or different, are junction groups capable of forming at least three H bonds, and in at least one embodiment, at least 4 H bonds, for example, 4 H bonds;

the arms L, which may be identical or different, are divalent linker arms, including a single covalent bond, linking a junction group A to a group G₁;

x is an integer greater than or equal to 1, for example, ranging from 1 to 12, and in at least one embodiment, x is equal to 1 or 2;

y is an integer greater than or equal to 1, for example, ranging from 1 to 12, and in at least one embodiment, y is equal to 1 or 2;

z is an integer greater than or equal to 1, for example, ranging from 1 to 6, and in at least one embodiment, z is equal to 1;

G₂, which may be identical or different, are monomers free of a junction group A and comprising at least one (co)polymerizable group capable of forming a covalent bond with a (co)polymerizable group G₁ of a monomer (I) and/or with a (co)polymerizable group borne by another identical or different monomer G₂;

m is the number of moles of identical or different, homopolymerized or copolymerized monomers of formula (I) and is an integer ranging from 1 to 12, for example, ranging from 2 to 8, or from 2 to 6;

n is the number of moles of identical or different, homopolymerized or copolymerized monomers of formula (II), and is an integer ranging from 0 to 20 000, for instance, from 1 to 10 000, or from 1 to 5000; and m+n≧2, and in at least one embodiment, m+n≧4;

wherein at least one of the groups A and/or groups G₁ and/or arms L and/or monomers G₂ comprises at least one ionizable group.

It is to be understood that, in the identical or different monomers of formula (I), when they comprise several groups G₁ and/or several linker arms L and/or several junction groups A, they may be identical or different.

In at least one embodiment, the (co)polymers according to the present disclosure may be formed by reacting at least one monomer of formula G₁-L-A and/or G₁-L-A-L-G₁ and/or G₁-L(A)-G₁ with at least one monomer of formula G₂, wherein the groups G₁, the linker arms L, the junction groups A, and the monomers G₂ may be identical or different and are defined as above.

In this definition, G₁-L(A)-G₁ denotes monomers in which the junction group A is linked via only one end to the linker group L and G₁-L-A-L-G₁ denotes monomers in which the junction group A is linked to two identical or different linker arms L, at its two ends.

When the (co)polymers according to the present disclosure are formed by reacting a mixture of monomers (I) and (II), according to one embodiment, they may result from the copolymerization of at least two moles of monomers (I) and of at least 1 mole of monomer (II).

In the case of the (co)polymers resulting from the homo- or copolymerization of monomers (I), according to another embodiment, these (co)polymers may comprise one mole of junction group (A) per monomer unit.

In the case of copolymerization of a mixture of monomers of formula (I) or of copolymerization of a mixture of monomers of formula (I) and of formula (II), in at least one embodiment, these copolymers may comprise at least two moles of junction groups (A), for example, 4 or 6 moles of junction groups (A) per copolymer chain.

According to yet another embodiment, the (co)polymers may have a polymer backbone with a degree of polymerization ranging from 2 to 20 000, for example, from 5 to 10 000, or from 10 to 5000.

In a further embodiment, the number-average molecular mass (Mn) of the (co)polymers according to the present disclosure may range from 1000 to 3 000 000, for instance, from 5000 to 1 000 000, or from 8000 to 500 000.

It has been found that the use of such (co)polymers, either alone or as a mixture with (co)polymers according to the present disclosure, comprising at least one junction group, in a cosmetic composition, may lead, after application of the composition to keratin materials, either to the formation of a supramolecular (co)polymer in the form of a physically crosslinked three-dimensional network, or to the formation of a linear supramolecular (co)polymer in the form of a long polymer chain, generally of high molecular mass, resulting from the physical connection of the (co)polymers of the present disclosure with each other.

General Structure of the (Co)Polymers According to the Present Disclosure

The (co)polymers according to the present disclosure may be chosen, for example, from the following structures:

linear (co)polymers α,ω-functionalized with junction groups (A);

linear (co)polymers comprising more than two junction groups, located in the chain and/or at one or both ends and/or as branches; and/or

branched (co)polymers with junction groups in the chain and/or as branches and/or at one or both ends.

It is to be understood that the (co)polymers according to the present disclosure may have only one of these structures, or may have a mixture of these structures, in all proportions.

According to at least one embodiment, the (co)polymers may be chosen from (co)polymers comprising at least 2 junction groups, for example, 4 junction groups, at least 4 junction groups, 6 junction groups, or at least 6 junction groups.

General Definition of the Junction Groups A

According to the present disclosure, a junction group A is a chemical, for instance, a carbon-based group capable of forming at least three H bonds, and in at least one embodiment, at least 4 H bonds, for example, 4 H bonds, and optionally comprising at least 3 identical or different heteroatoms, and in at least one embodiment, at least 4 heteroatoms, such as 4 heteroatoms, which may be identical or different, chosen from O, N, S, P, and F, and in at least one embodiment, from O, S, and N.

These junction groups may comprise, for example, at least 3 functional groups, in at least one embodiment, at least 4 groups, for example, 4 functional groups, chosen from:

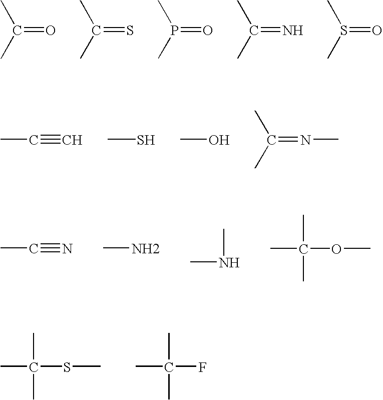

These functional groups may be classified into two categories:

H-bond-donor functional groups such as the groups:

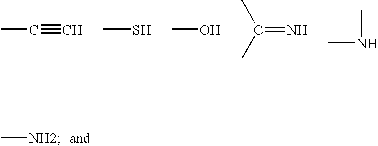

H-bond-acceptor functional groups such as the groups:

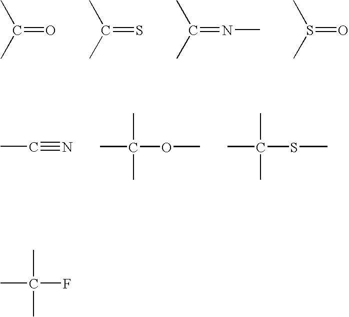

The junction groups A form a basic structural element comprising at least 3 groups, and in at least one embodiment, at least 4 groups, for example, 4 groups, capable of establishing H bonds.

The basic structural elements capable of establishing 3 or 4 H bonds may be represented schematically in the following manner: 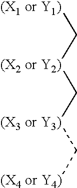
wherein X₁ is an H-bond-acceptor functional group and Y₁ is an H-bond-donor functional group.

Thus, each structural element should be able to establish H bonds with at least one partner structural element, which may be identical (i.e. self-complementary) or different, such that each pairing of two partner structural elements takes place by formation of at least three H bonds, and in at least one embodiment, at least 4 H bonds, for example, 4 H bonds. A proton acceptor X will become paired with a proton donor Y.

Several possibilities are offered, for example:

| | | |
|---|---|---|
| pairing of: | XXXX with YYYY: | |
| | XXXY with YYYX: | |
| | XXYX with YYXY: | |
| | XYYX with YXXY: | |
| | XXYY with YYXX | self-complementary or otherwise; |
| | XYXY with YXYX | self-complementary or otherwise. |

According to one embodiment, the junction groups A can establish 4 H bonds with an identical (or self-complementary) partner group, among which 2 are donor bonds (for example NH) and 2 are acceptor bonds (for example CO and —C═N—).

In another embodiment, the junction groups comprise 5- or 6-atom rings (for example, aromatic rings and unsaturated heterocycles) comprising C and/or N atoms with conjugated double bonds to stabilize and direct the H interactions.

According to a further embodiment, the junction groups are engaged in 6-atom rings comprising C and/or N with conjugated double bonds to stabilize and direct the H interactions.

In yet another embodiment, the junction groups (A) are capable of establishing at least four H bonds with the same partner junction group (self-complementary).

The junction groups (A) capable of forming 3 or 4 H bonds may be chosen from the following families, it being understood that all tautomeric forms are included:

(i) aminopyrimidones of formula:

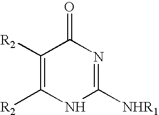

(ii) ureidopyrimidones of formula:

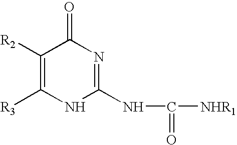

(iii) acylaminopyridines, for example:
monoacylaminopyridines of structure: 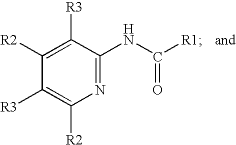
di(acylamino)pyridines, such as 2,6-di(acylamino)pyridines of structure:

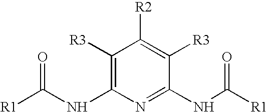

(iv) aminopyrimidines, for example:
aminopyrimidine compounds: 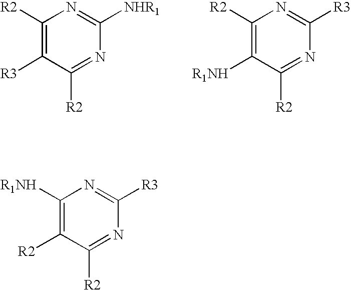
diaminopyrimidine compounds: 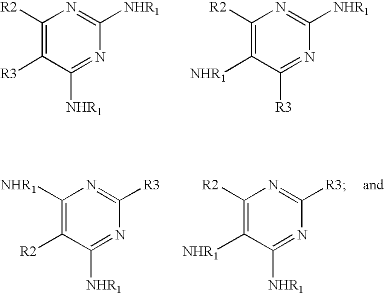
triaminopyrimidine compounds;

(v) ureidotriazines, for instance, the mono-, di-, and triureidotriazines, such as ureidoaminotriazines of structure:

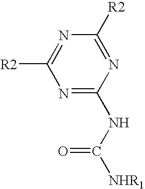

(vi) (acylamino)triazines, for instance, the mono-, di-, and triacylaminotriazines, optionally amino (mono-, di- or triamino), such as:
di(acylamino)triazines of structure: 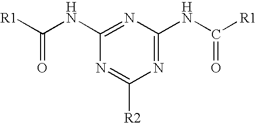
acylamino, aminotriazines (mono- or diacylamino, and mono- or diamino), for example compounds of the structure: 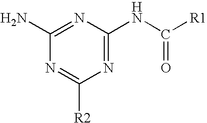
acylaminotriazines of structure: 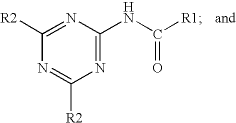
triacylaminotriazines,

(vii) aminotriazines including, but not limited to:
monoaminotriazines,
2,6-diamino-s-triazines of structure: 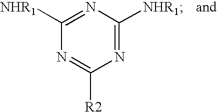
triamino-s-triazine compounds of structure:

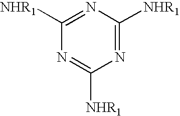

(viii) acylaminotriazoles of structure:

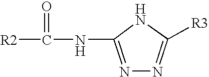

(ix) compounds of the urazoylbenzoic acid family of structure:

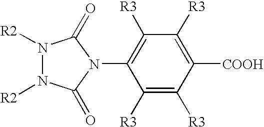

(x) phthalhydrazides of structure:

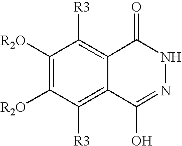

(xi) uracils of structure:

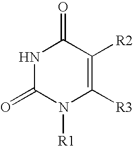

(xii) thymines of structure:

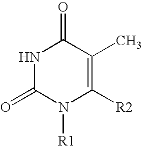

(xiii) succinimides of structure:

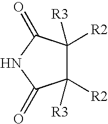

(xiv) glutarimides of structure:

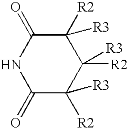

(xv) compounds of the cyanuric acid family of structure:

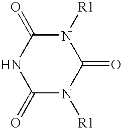

(xvi) maleimides:

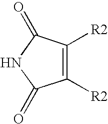

(xvii) compounds of the barbituric acid family of structure:

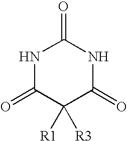

(xviii) compounds of structures:

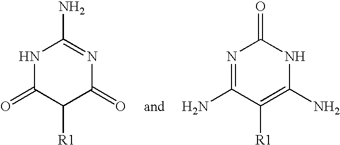

(xix) compounds of the trimellitic family, of formula:

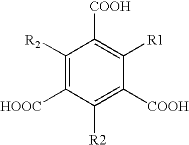

(xx) ureidopyridines, for example, mono- or diureidopyridines, such as those of formulas:

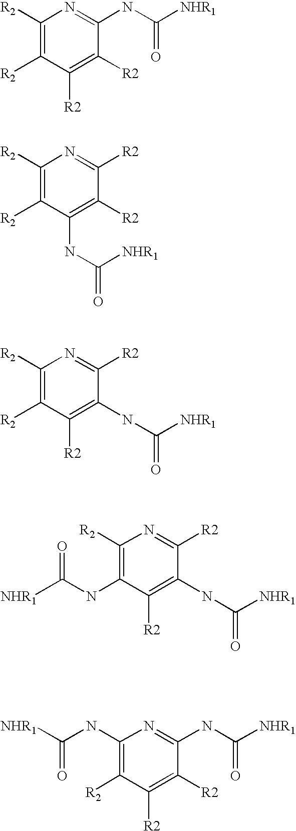

(xxi) carbamoylpyridines of formulas:

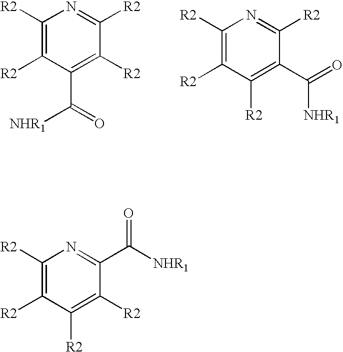

(xxii) adenines of formula:

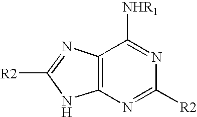

(xxiii) guanines of formula:

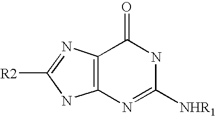

(xxiv) cytidines of formula:

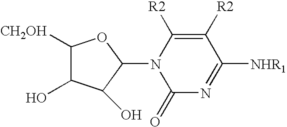

In all the formulas given above in (i)-(xxiv), the radicals have the following meanings:

(a) the radicals R¹, which may be identical or different, are chosen from H, halogen atoms, and/or monovalent linear, branched, or cyclic, saturated or unsaturated, optionally aromatic, C₁-C₆₀₀₀ carbon-based groups (for example, alkyl), which may comprise at least one heteroatom such as O, S, N, P, Cl, Br, and F; or a combination of these meanings.

According to one embodiment, the radical R¹ may be chosen from C₄-C₁₂ cycloalkyl groups; linear or branched C₁-C₃₀ alkyl groups, and C₄-C₁₂ aryl groups; optionally substituted with at least one function chosen from amino, ester, and/or hydroxyl functions.

In another embodiment, R₁ is chosen from: —C₄H₉, -phenyl; 1,4-nitrophenyl, 1,2-ethylene, 1,6-hexylene, 1,4-butylene, 1,6-(2,4,4-trimethylhexylene), 1,4-(4-methylpentylene), 1,5-(5-methylhexylene), 1,6-(6-methylheptylene), 1,5-(2,2,5-trimethylhexylene), 1,7-(3,7-dimethyloctylene); -isophorone-, 4,4′-methylenebiscyclohexylene, tolylene, 2-methyl-1,3-phenylene, 4-methyl-1,3-phenylene, and 4,4-biphenylenemethylene groups. According to a further embodiment, R¹ is chosen from: -isophorone-, —(CH₂)₂—, —(CH₂)₆—, —CH₂CH(CH₃)—CH₂—C(CH₃)₂—CH₂—CH₂, 4,4′-methylenebis(cyclohexylene), and 2-methyl-1,3-phenylene groups.

(b) the radicals R², which may be identical or different within the same formula, are chosen from H, halogen (for example, —Br, —Cl, or —F), —OH, —N(R)₂ (wherein R is chosen from H and linear or branched C₁-C₁₂, for example, C₁-C₄, alkyl radicals, such as methyl and ethyl radicals); and monovalent linear, branched or cyclic, saturated or unsaturated, optionally aromatic, C₁-C₆₀₀₀ hydrocarbon-based groups, which may comprise at least one heteroatom such as O, S, N, P, and F; or a combination of these meanings;

According to one embodiment, the radicals R² may be chosen from

—CN,

—NH₂,

C₁-C₃₀ alkyl groups;

C₄-C₁₂ cycloalkyl groups;

C₄-C₁₂ aryl groups;

(C₄-C₁₂)aryl(C₁-C₃₀)alkyl groups;

C₁-C₄ alkoxy groups;

arylalkoxy groups, such as (C₁-C₄) arylalkoxy groups;

C₄-C₁₂ heterocycles;

thioalkoxy groups,

sulfoxy groups, and

mixtures thereof,

these groups being optionally substituted with at least one function chosen from amino, ester, and/or hydroxyl functions.

In another embodiment, R² is chosen from H, CH₃, C₁₃H₂₇, C₇H₁₅, and phenyl.

(c) the radicals R³, which may be identical or different within the same formula, are chosen from H and monovalent linear, branched or cyclic, saturated or unsaturated, optionally aromatic, C₁-C₆₀₀₀ hydrocarbon-based groups, which may comprise at least one heteroatom such as O, S, N, P, and F; or a combination of these meanings;

According to yet another embodiment, the radical R³ may be chosen from C₄-C₁₂ cycloalkyl groups; linear or branched C₁-C₃₀ alkyl groups, and C₄-C₁₂ aryl groups; optionally substituted with at least one function chosen from amino, ester, and/or hydroxyl functions.

In all these formulas, it is to be understood that at least one, for example, one or two, of the groups R¹ and/or R² is the point of attachment of the junction group A to the polymer backbone POL.

In at least one embodiment, the point of attachment is borne by R¹ and/or R², and when there is only one point of attachment, it is borne by the group R¹.

The junction groups (A) may be chosen, for example, from:

(a) complementary and identical, i.e. self-complementary, junction groups (A), such as:
aminopyrimidones and ureidopyrimidones,
compounds of the trimellitic acid family and compounds derived from urazoylbenzoic acid,
acylaminopyridines, ureidopyridines, and carbamoylpyridines,
acylaminotriazines, ureidotriazines, ureidoaminotriazines, and diaminotriazines,
acylaminotriazoles,
phthalhydrazides, and
compounds of formulas: 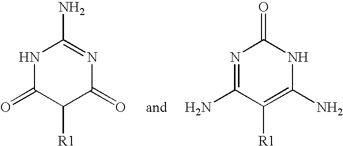
   wherein R₁ is chosen from H and linear, branched, or cyclic, saturated or unsaturated, optionally aromatic, C₁-C₆₀₀₀ hydrocarbon-based groups, which may comprise at least one heteroatom such as O, S, N, P, and F.

(b) complementary but different junction groups (A), for example:
adenine, which is complementary to guanine,
cytidine, which is complementary to thymine,
triamino-s-triazine, which is complementary to uracil, succinimide, glutarimide, cyanuric acid, thymine, maleimide, (di)aminopyrimidine, and barbituric acid; and
acylaminoamino-s-triazine, which is complementary to uracil, succinimide, glutarimide, cyanuric acid, thymine, maleimide, (di)aminopyrimidine, and barbituric acid.

In at least one embodiment, the junction groups A are chosen from groups which are capable of establishing at least three H bonds with each other (self-complementary), for instance, at least four H bonds with each other. These groups include, but are not limited to:

ureidopyrimidones;

ureidopyridines and carbamoylpyridines;

acylamino-s-triazines, such as acyldiamino-s-triazines;

ureidotriazines;

phthal hydrazides; and

compounds of formulas: 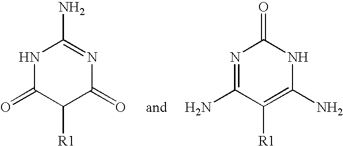
wherein the radicals R¹, R², and R³ have the meanings given above.

According to another embodiment, the junction groups capable of establishing at least three H bonds with each other, may be chosen, for instance, from the following groups:

2-ureidopyrimidone;

6-methyl-2-ureidopyrimidone;

diacyl-2,6-diamino-s-triazine;

ureido-s-triazine; and

compounds of formulas: 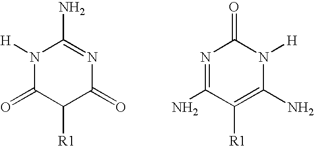
wherein the radicals R¹, R², and R³ have the meanings given above.

As indicated previously, the junction groups A may be linked to the (co)polymerizable groups G₁ via at least one linker arm L, and in at least one embodiment, only one linker arm L.

The linker arm L may be a single covalent bond.

The linker arm L may be formed during the reaction:

either between a reactive function linked to the junction group A with a reactive function borne by the (co)polymerizable group G₁;

or between a reactive function linked to a precursor of the junction group A with a reactive function borne by the (co)polymerizable group G₁ to form simultaneously the junction group A and the species A-L-G₁;

wherein the two reactive functions are capable of reacting together, and are optionally linked directly or via a divalent segment to the junction group A and/or to the group G₁, and/or to the precursor of the group A.

As used herein, the term “A” means the junction group without its reactive function.

The reactive functions may be chosen, for instance, from the following functions:

isocyanate —N═C═O;

isothiocyanate —N═C═S;

carboxylic acid or ester —COOR, wherein R is chosen from H and linear or branched C₁-C₁₂, for example, C₁-C₄, alkyl radicals, such as methyl and ethyl radicals;

hydroxyl-OH;

primary, secondary, or tertiary amines —N(R)₂, wherein R, which may be identical or different, is as defined above;

ethylenic —CR═C(R)₂, wherein R, which may be identical or different, is as defined above;

a function chosen from: 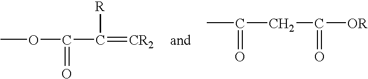
wherein R, which may be identical or different, is as defined above; and

the salified and activated forms thereof; for example, the reactive group OH may be activated in the form of O-tosylate; the carboxylic acid group may be activated in the form of acid chloride or acid anhydride; activations with carbonyldiimidazole may also be mentioned, such as:

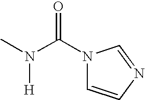

According to one embodiment, the reactive function that is a precursor of the linker arm L may be chosen from isocyanate, hydroxyl, and amine functions, and in at least one embodiment, an isocyanate function.

When the reactive function is linked via a divalent segment to A and/or G₁ and/or to the precursor of A, this segment may be chosen from:

linear or branched, optionally substituted alkylene radicals;

optionally substituted cycloalkylene radicals;

optionally substituted arylene radicals;

amino (—NH— or —NR—), —O—, —SO—, —SO₂—, and —C(O)— radicals;

and also combinations thereof of the same category and/or of different categories, and in at least one embodiment, those leading to radicals chosen from cycloalkylenealkylene, biscycloalkylene, biscycloalkylenealkylene, arylenealkylene, bisphenylenealkylene, oxyalkylene, and aminoalkylene radicals.

These radicals may optionally be substituted, for example, with at least one C₁-C₁₂ alkyl group optionally comprising heteroatoms chosen from O, N, S, F, and P, and combinations thereof.

Non-limiting examples of suitable optionally substituted alkylene radicals include C₁-C₃₀ alkylene radicals, for example, methylene radicals —CH₂— and poly(methylene) radicals of formula —(CH₂)ₙ— (wherein n≧2), such as ethylene, butylene, 1,4-butylene, and 1,6-hexylene radicals and branched C₃-C₁₀ alkylene radicals such as 1,4-(4-methylpentylene), 1,6-(2,2,4-trimethylhexylene), 1,5-(5-methylhexylene), 1,6-(6-methylheptylene), 1,5-(2,2,5-trimethylhexylene), 1,7-(3,7-dimethyloctylene), 2,2-(dimethylpropylene), and 1,6-(2,4,4-trimethylhexylene) radicals.

Examples of optionally substituted cycloalkylene radicals include, but are not limited to, cyclopentylene and cyclohexylene radicals, optionally substituted, for instance, with alkyl groups.

A non-limiting example of a suitable cycloalkylenealkylene is the isophorone radical of formula:

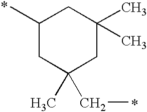

Suitable optionally substituted biscycloalkylenealkylene radicals may include, but are not limited to radicals of formula: 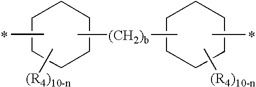
wherein b is an integer ranging from 0 to 3, n is an integer ranging from 0 to 4; and R₄, which may be identical or different, is chosen from H and C₁-C₁₂ alkyl radicals, such as methyl radicals; for instance, the optionally substituted biscycloalkylenealkylene may be 4,4′-methylenebis(cyclohexylene).

Examples of optionally substituted arylene radicals include, but are not limited to, phenylene radicals, tolylene radicals, such as 2,4- and 2,6-tolylene radicals, and naphthylene radicals, such as 2,4-naphthylene and 2,6-naphthylene.

The optionally substituted arylenealkylene radicals may be chosen, for example, from phenylenealkylene radicals such as the benzylene radical: 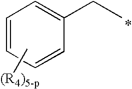
wherein p is an integer ranging from 0 to 5.

Non-limiting examples of optionally substituted bis(phenylene)alkylene radicals may include: (i) the radicals of formula: 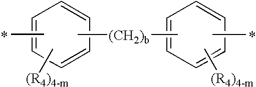
wherein b is an integer ranging from 0 to 3 and m is an integer ranging from 0 to 4; such as the bis(phenylene) radical and the 4,4′-methylenebis(phenylene) radical, and (ii) the radicals of formula: 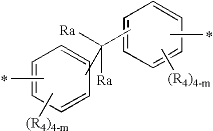
wherein m is an integer ranging from 0 to 4 and the radicals Rₐ, which may be identical or different, are chosen from H and C₁-C₄ alkyl radicals, such as methyl radicals.

Suitable optionally substituted oxyalkylene radicals may include, for example, the alkylene oxide radicals of formula O—(R′O)_{y}— wherein R′, which may be identical or different, is chosen from linear or branched C₂-C₄ alkylene radicals, such as ethylene and propylene; and y is an integer ranging from 1 to 500, for example, from 1 to 200, or from 5 to 100.

In one embodiment of the present disclosure, A may bear at least one ionizable group as defined below. For instance, at least one of the radicals R¹, R², and/or R³ may comprise at least one ionizable group.

Definition of the (Co)Polymerizable Groups G₁

The (co)polymerizable group G₁ is a chemical group capable of reacting with an identical or different chemical group borne by another group G₁ of another identical or different monomer (I), and/or with a chemical group of a monomer (II) of formula G₂, to form a (co)polymer comprising at least two repeating units.

As used herein, the term “comprising at least two repeating units” means a constituent unit of a homopolymer or copolymer resulting from the homopolymerization or copolymerization of at least two identical or different monomer or oligomer units.

The homopolymerizable and/or copolymerizable functions of the groups G₁ may be chosen, for example, from functions that can undergo at least one (co)polymerization mechanism chosen from radical (co)polymerization anionic (co)polymerization cationic (co)polymerization, (co)polymerization by polyaddition, (co)polymerization by polycondensation, (co)polymerization by ring opening, and any other (co)polymerization mechanism.

Functions that can undergo a radical, anionic, or cationic (co)polymerization include, for instance, activated or unactivated ethylenic double bonds, such as olefinic functions, vinyl functions, allylic functions, (meth)acrylic functions, (meth)acrylamide functions, and combinations thereof.

Examples of functions that may be polymerized by polyaddition or by polycondensation include, but are not limited to, hydroxyl, primary and secondary amine, ester, carboxylic acid, and isocyanate functions, which may or may not be activated.

Suitable functions that may be polymerized by anionic or cationic ring opening, may include, for example, cyclic esters, cyclic amides, cyclic carbonates, and cyclic ethers.

Mention may also be made of reactions between halides and tertiary amines.

In one embodiment of the present disclosure, G₁ may bear at least one ionizable group as defined below.

Definition of the Linker Arms L

The linker arms L, which may be identical or different, are in general divalent groups linking the junction groups (A) to the (co)polymerizable groups G₁.

These linker arms may be a single covalent bond.

They may also be saturated or unsaturated, linear, branched or cyclic (including aromatic) divalent carbon-based groups, such as alkyls, and also combinations thereof, comprising from 1 to 6000, for example, from 1 to 30 carbon atoms, and possibly comprising at least one, for instance, from 1 to 5, identical or different heteroatom chosen from O, N, S, P, and F, and in at least one embodiment, O, N, and S.

When it is present, the at least one heteroatom may be in the chain of the linker arm L or may participate in a substituent group on the chain of the said linker arm, for example, hydroxyl, ester, thiol, amine groups (NR₂ wherein R, which may be identical or different, is chosen from linear C₁-C₁₂, for example, C₁-C₄, alkyl radicals, such as methyl and ethyl groups), and PEO (polyethylene oxide).

In general, the linker arms L may be chosen, for example, from C₁-C₃₀ carbon-based groups, such as alkylenes (divalent alkyls), which may comprise functional groups chosen from:

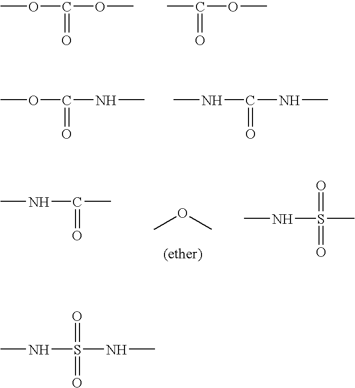

Thus, the groups G-L-A-L-G may be of the type: 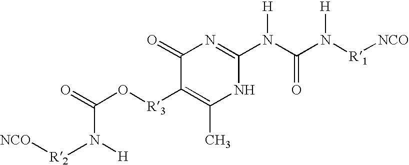
and the groups A-L-G may be of the type: 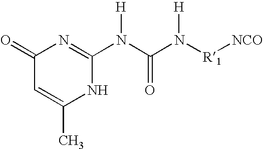
wherein the radicals R′₁, R′₂ and R′₃, which may be identical or different, are chosen from divalent carbon-based groups, including but not limited to, 1,2-ethylene, 1,6-hexylene, 1,4-butylene, 1,6-(2,4,4-trimethylhexylene), 1,4-(4-methylpentylene), 1,5-(5-methylhexylene), 1,6-(6-methylheptylene), 1,5-(2,2,5-trimethylhexylene), 1,7-(3,7-dimethyloctylene); -isophorone-, 4,4′-methylenebis(cyclohexylene), tolylene, 2-methyl-1,3-phenylene, 4-methyl-1,3-phenylene, and 4,4-bis(phenylene)methylene.

In at least one embodiment, the radicals R′₁, R′₂ and R′₃, independently of each other, may be chosen from isophorone, —(CH₂)₂—, —(CH₂)₆—, —CH₂CH(CH₃)—CH₂—C(CH₃)₂—CH₂—CH₂, 4,4′-methylenebis(cyclohexylene), and 2-methyl-1,3-phenylene.

According to another embodiment, R′₁=R′₂=-isophorone- and R′₃=—(CH₂)₂—.

In yet another embodiment, the group G-L-A-L-G may have the following formula:

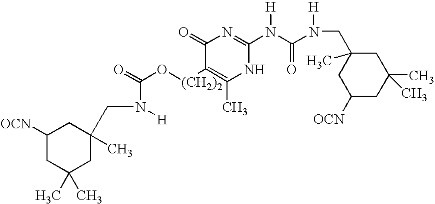

The (co)polymerizable groups G₁ may be divided into three major classes, namely:

(co)polymerizable groups comprising at least one ethylenic double bond capable of undergoing radical, anionic, or cationic (co)polymerization, for example, CH₂═C(CH₃)— and CH₂═CH— groups;

groups that may be (co)polymerized via nucleophilic or electrophilic substitution or addition reaction, or radical addition, and in at least one embodiment, via polyaddition or polycondensation, chosen, for example, from groups comprising at least one function chosen from hydroxyl (OH) functions, activated hydroxyl functions such as tosylate, thiol (SH) functions, halide (e.g., Br, Cl) functions, primary or secondary amine (e.g., NH₂ and NHR) functions, ester (COOR) functions, carboxylic acid (COOH) functions, activated acid functions such as COHal, protected or unprotected isocyanate (NCO) functions, isothiocyanate (NCS) functions, —C═C—, —C(O)H functions, —SiH functions, succinimide functions, oxazoline functions, acetal functions, hemiacetal functions, chlorotriazine functions, —SO₂Cl functions, and epoxide functions; wherein the radical R is chosen from C₁-C₆ alkyl radicals, such as methyl; and

groups that may be (co)polymerized via anionic or cationic ring opening, such as groups comprising at least one group chosen from cyclic ether, cyclic ester, cyclic amide, and cyclic carbonate groups.

Monomers of formula (I) may be chosen, for example, from:

i) monomers that may undergo radical, anionic, or cationic (co)polymerization, which may be represented by the formula: 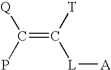
wherein P, Q, and T, which may be identical or different, are chosen from hydrogen, linear or branched, cyclic or non-cyclic, saturated or unsaturated, or aromatic, hydrocarbon-based radicals, such as alkyl radicals, comprising from 1 to 12 carbon atoms, and groups -L-A, L; and A is defined above.

In at least one embodiment, the monomers (I) comprise 1 or 2 groups -L-A.

Examples of such monomers that may undergo a radical, anionic, or cationic (co)polymerization include, but are not limited to, monomers of formula: 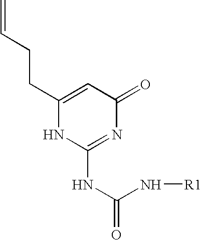
wherein R₁ is as defined above,
and also the acrylate derived from 6-methyl-2-ureidopyrimidone of formula:

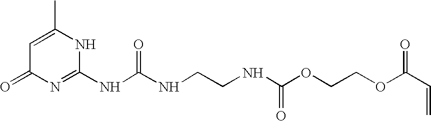

These monomers may be prepared, for instance, according to the method described in International Patent Application Publication No. WO 98/14504.

Other examples of suitable monomers include those of formulas: 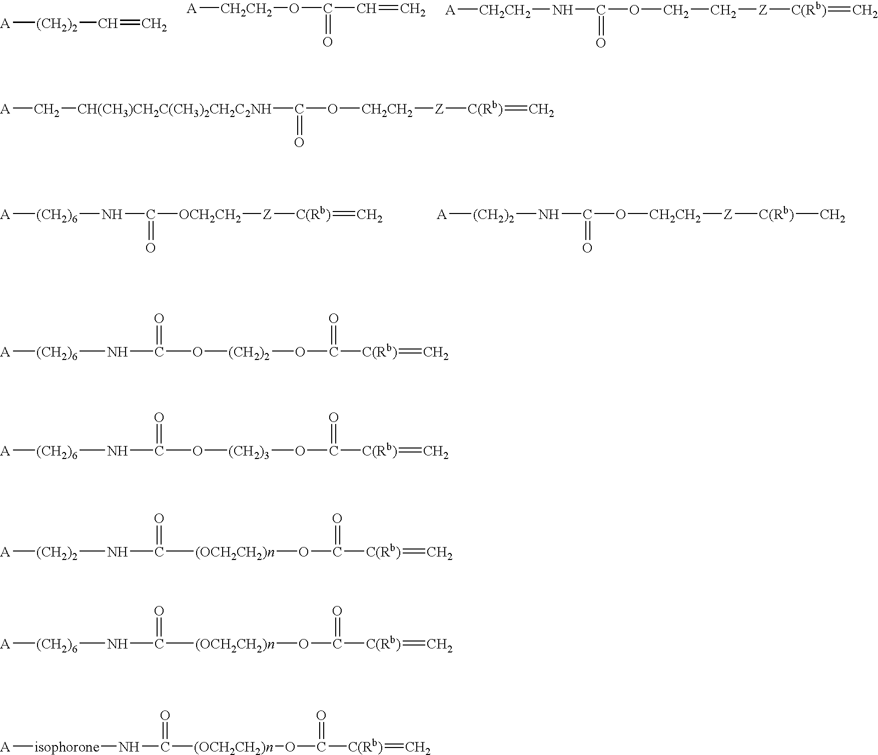
wherein:

Z is chosen from —O—C(O)— and —NH—C(O)—;

n is an integer ranging from 1 to 500;

R^{b} is chosen from H and CH₃; and

A is chosen from groups of formula: 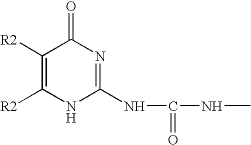
wherein the groups R₂, which may be identical or different, are defined above.

These monomers may be prepared, for example, according to the method described in U.S. Patent Application Publication No. US 2004/0034190.

ii) monomers that may be (co)polymerized via addition or polycondensation, comprising at least one group G₁, and in at least one embodiment, two groups G₁, which may (co)polymerize via addition or polycondensation.

These groups G₁ may be identical or different on the same monomer and may be copolymerizable with a neighboring monomer, it being understood that a person skilled in the art will select the monomers taking their reactivity into account.

The (co)polymerizable groups may be chosen from the reactive functions mentioned above.

The (co)polymers resulting from the polyaddition or polycondensation may then be chosen from polyurethanes, polyureas, aliphatic or aromatic polyesters, aliphatic or aromatic polyamides, and copolymers thereof, for instance, poly(urethane/ureas) and poly(ester/amides).

In one embodiment, the monomers may comprise 1 or 2 groups chosen from A-L- or -L-A-L-, and in another embodiment, the monomers may comprise one group chosen from A-L- and -L-A-L-.

Examples of such monomers that may be (co)polymerized via polyaddition or polycondensation include, but are not limited to, monomers of formulas:

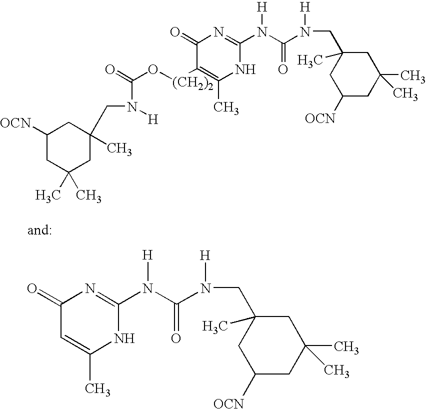

iii) monomers that (co)polymerize via ring opening.

These monomers may comprise groups G₁ that may be chosen from:

cyclic ethers of formula: 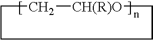
wherein R is chosen from H and linear or branched, cyclic or non-cyclic, saturated or unsaturated, or aromatic, hydrocarbon-based radicals, such as C₁-C₁₂ alkyls, and n is an integer ranging from 1 to 3. In at least one embodiment R is chosen from H and CH₃ and n=1;

cyclic amides of formula: 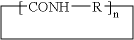
wherein which R is —(CH₂)ₘ— and m is an integer ranging from 3 to 12, and in at least one embodiment, equal to 3 or 5, and n is an integer ranging from 1 to 3, and in at least one embodiment, equal to 1;

cyclic esters of formula:

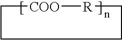

wherein R is —(CH₂)ₘ— and m is an integer ranging from 3 to 12, and in at least one embodiment, equal to 3 or 5, and n is an integer ranging from 1 to 3, and in at least one embodiment, equal to 1;

cyclic carbonates of formula: 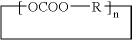
wherein R is —(CH₂)ₘ— and m is an integer ranging from 3 to 12, and in at least one embodiment, equal to 3 or 5, and n is an integer ranging from 1 to 3, and in at least one embodiment, equal to 1;

cyclic perfluoroethers, lactides, oxazolines, norbonene, and derivatives thereof; and

combinations thereof.

The (co)polymers resulting from the (co)polymerization of cyclic ethers may include, but are not limited to, poly(oxyalkylenes), such as poly(oxyethylenes) and poly(oxypropylenes) and copolymers thereof, such as poly(oxyethylene/oxypropylenes).

Polyamides resulting from such polymerizations include, for example, polycaprolactams and polypyrrolidones.

Non-limiting examples of polyesters resulting from such polymerizations include polycaprolactones.

Examples of polyoxazolines resulting from such polymerizations include, but are not limited to, poly(2-methyloxazoline) and poly(2-ethyloxazoline).

Poly(norbornenes) and derivatives thereof may also be obtained.

In one embodiment of the present disclosure, L, when it is not a single bond, may bear at least one ionizable group as defined below.

Definition of the Monomers (II) of Formula G₂

The monomers (II) G₂ may be chosen as a function of the intended (co)polymerization mechanism to be able to (co)polymerize with the groups G₁ of the monomers (I).

One exemplary class of monomers (II) G₂ includes, but is not limited to, ethylenic monomers, i.e. monomers comprising a reactive double bond capable of reacting under radical, anionic, or cationic conditions.

Non-limiting examples of suitable ethylenic monomers include:

i) (meth)acrylates chosen from those of formulas CH₂═CHCOOR⁴ and CH₂═C(CH₃)COOR⁴, wherein R⁴ is chosen from:
hydrogen,
linear, cyclic or branched alkyl groups (such as cycloalkyl and alkylcycloalkyl) comprising from 1 to 30 carbon atoms, in which is optionally intercalated at least one heteroatom chosen from O, N, S, and P, the alkyl group also possibly being substituted with at least one substituent chosen from hydroxyl groups, halogen atoms (Cl, Br, I, and F), and groups Si(R₇)(R₈) wherein R₇ and R₈, which may be identical or different, are chosen from C₁-C₆ alkyl groups and phenyl groups;
C₃ to C₂₀ aryl groups such as phenyl groups;
C₄ to C₃₀ aralkyl and alkylaryl groups, wherein the alkyl group is chosen from C₁ to C₈ alkyl groups, such as 2-phenylethyl and benzyl groups;
C₄-C₁₂ heterocycloalkyl groups containing at least one heteroatom chosen from O, N, P, and S, the ring being aromatic or non-aromatic; such as imidazole;
C₄-C₃₀ alkylheterocycloalkyl groups, wherein the alkyl group is chosen from C₁-C₈ alkyl groups, such as furfurylmethyl and tetrahydrofurfurylmethyl,
   wherein the said aryl and aralkyl groups may optionally comprise at least one intercalated heteroatom chosen from O, N, S, and P, and/or may be substituted with at least one substituent chosen from hydroxyl groups, halogen atoms, and linear or branched C₁-C₄ alkyl groups, which may themselves comprise at least one intercalated heteroatom chosen from O, N, S, and P and/or which may be substituted with at least one substituent chosen from hydroxyl groups, halogen atoms (Cl, Br, I, and F) and groups Si(R₇)(R₈), wherein R₇ and R₈, which may be identical or different, are chosen from C₁ to C₆ alkyl groups and phenyl groups.

In at least one embodiment, the radicals R⁴ may be chosen, for example, from methyl, ethyl, propyl, n-butyl, isobutyl, tert-butyl, hexyl, ethylhexyl, octyl, lauryl, stearyl, isooctyl, isodecyl, and dodecyl groups; alkyl-based groups (i.e. substituted and/or interrupted alkyls) such as C₁-C₄ hydroxyalkyl groups such as 2-hydroxyethyl, 2-hydroxybutyl and 2-hydroxypropyl; (C₁₋₄)alkoxy(C₁₋₄)alkyl groups such as methoxyethyl, ethoxyethyl, and methoxypropyl; C₃ to C₁₂ cycloalkyl groups such as isobornyl, t-butylcyclohexyl, and cyclohexyl; and t-butylbenzyl, phenyl, furfurylmethyl, tetrahydrofurfurylmethyl, and 2-ethylperfluorohexyl groups.

R⁴ may also be chosen from groups —(OC₂H₄)q-OR, wherein q=5 to 500 and R is chosen from H and C₁ to C₃₀ alkyl groups, for example -PEO-methoxy and -PEO-behenyl.

ii) (meth)acrylamides of formulas CH₂═CHCONR⁶R⁵ and CH₂═C(CH₃)CONR⁶R⁵, wherein R⁵ and R⁶, which may be identical or different, have the same meanings as for the groups R⁴ above.

Examples of (meth)acrylamide monomers include, but are not limited to, (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylcacrylamide, undecylacrylamide, and N-(2-hydroxypropylmethacrylamide).

iii) vinyl monomers chosen from those of formulas: CH₂═CH—R⁹, CH₂═CH—CH₂—R⁹, and CH₂═C(CH₃)—CH₂—R⁹ wherein R⁹ is a group chosen from hydroxyl, halogen (Cl and F), NH₂, acetamide (—NHCOCH₃), —OR₁₀ wherein R₁₀ represents is chosen from phenyl groups and C₁-C₁₂ alkyl groups (vinyl ether); —OCOR₁₁ (vinyl ester) wherein R₁₁ is chosen from:

(i) linear or branched C₂ to C₁₂ alkyl groups,

(ii) C₃ to C₁₂ cycloalkyl groups such as isobornyl and cyclohexyl,

(iii) C₃ to C₂₀ aryl groups such as phenyl,

(iv) C₄ to C₃₀ aralkyl groups (wherein the alkyl group is chosen from C₁ to C₈ alkyl groups) such as 2-phenylethyl and benzyl,

(v) saturated or unsaturated, aromatic or non-aromatic, 4- to 12-membered heterocycloalkyl groups containing at least one heteroatom chosen from O, N, and S, such as furfuryl and tetrahydrofurfuryl, and

(vi) C₁ to C₄ alkylheterocycloalkyl groups, such as furfurylmethyl and tetrahydrofurfuryl methyl,

wherein the alkyl, cycloalkyl, aryl, aralkyl, heterocycloalkyl, and alkylheterocycloalkyl groups may be optionally substituted with at least one substituent chosen from hydroxyl groups, halogen atoms, and linear or branched C₁ to C₄ alkyl groups in which is optionally intercalated at least one heteroatom chosen from O, N, S, and P, the alkyl groups also possibly being substituted with at least one substituent chosen from hydroxyl groups, halogen atoms (Cl, Br, I, and F), and groups Si(R₇)(R₈) wherein R₇ and R₈, which may be identical or different, are chosen from C₁ to C₆ alkyl groups and phenyl groups.

Examples of vinyl monomers include, but are not limited to, vinylcyclohexane, styrene, N-vinylpyrrolidone, and N-vinylcaprolactam.

Non-limiting examples of vinyl esters include vinylacetate, vinylpropionate, vinylbutyrate, vinyl ethylhexanoate, vinyl neononanoate, and vinyl neododecanoate.

Suitable vinyl ethers may include, for example, methyl vinyl ether, ethyl vinyl ether, and isobutyl vinyl ether.

iv) other monomers such as (meth)acrylate, (meth)acrylamide, and vinyl monomers containing at least one group chosen from fluoro groups and perfluoro groups, such as ethylperfluorooctyl methacrylate, and silicone (meth)acrylic, (meth)acrylamide, and vinyl monomers.

In one embodiment of the present disclosure, G₂ may bear at least one ionizable group as defined below.

Definition of the Ionizable Groups

According to the present disclosure, at least one of the groups G₁ of the monomers (I), of the linker arms L of the monomers (I), of the junction groups (A) of the monomers (I), and/or of the optional monomers (II) G₂, which are constituents of the final (co)polymer according to the present disclosure, bears at least one ionizable group.

In one embodiment, the at least one ionizable group may be borne by a linker arm L of the monomer (I) when L is not a single bond, and in another embodiment, by the monomer (II) G₂ when this monomer participates in the constitution of the (co)polymer.

As used herein, the term “ionizable group” means any group which, either by its intrinsic chemical nature, or as a function of the medium and/or of the pH of the medium in which it is present, may be in ionic form.

According to its chemical nature, the at least one ionizable group may be chosen from cationizable, anionizable, and amphoteric groups. This also includes tetra-N-substituted quaternary ammonium ionic groups.

The at least one ionizable group may be present in the (co)polymer in an amount ranging from 0.1% to 50% by weight relative to the total weight of the (co)polymer according to the present disclosure, for example, from 0.5% to 35% by weight, or from 1% to 15% by weight relative to the total weight of the polymer. Thus, the at least one monomer bearing the at least one ionizable group may be present in the (co)polymer in an amount ranging from 3% to 20% by weight, for example, from 6% to 17% by weight, or from 8% to 15% by weight, relative to the total weight of the final (co)polymer.

The at least one ionizable group may be chosen, for example, from:

i) anionizable groups, and salts thereof, such as groups comprising an acid function chosen from:
carboxylic radicals: —COOH,
sulfonic radicals: —SO₃H,
—OSO₃H radicals,
phosphonic radicals: —(O)P(OH)₂,
phosphoric radicals: —OP(O)(OH)₂, and
organic or mineral salified forms thereof.

Neutralization of the acid groups may be performed with at least one base chosen from mineral bases such as LiOH, NaOH, KOH, Ca(OH)₂, NH₄OH, Mg(OH)₂, and Zn(OH)₂; and organic bases such as primary, secondary, and tertiary alkylamines, for instance, triethylamine and butylamine. The primary, secondary, and tertiary alkylamines may comprise at least one atom chosen from nitrogen and oxygen and may thus comprise, for example, at least one alcohol function; for instance, 2-amino-2-methylpropanol, triethanolamine, and 2-dimethylaminopropanol, lysine, and 3-(dimethylamino)propylamine.

ii) cationizable groups, and salts thereof, such as groups comprising a function chosen from:

a) amine radicals of formula —N(R₁₅)(R₁₆) and organic or mineral salts thereof, wherein R₁₅ and R₁₆, which may be identical or different, are chosen from:

(i) hydrogen,

(ii) linear, branched, or cyclic, saturated or unsaturated, optionally aromatic alkyl groups comprising from 1 to 30 carbon atoms and optionally comprising from 1 to 10 heteroatoms chosen from O, N, S, and P, for instance, methyl, ethyl, propyl, isopropyl, n-butyl, t-butyl, isobutyl, lauryl, and stearyl groups;

(iii) alkylene oxide groups of formula —(R₁₇O)ᵣR₁₈ wherein R₁₇ is chosen from linear or branched C₂-C₄ alkyl radicals, R₁₈ is chosen from hydrogen and linear or branched C₁ to C₃₀ alkyl radicals and r is a number ranging from 1 to 250; or

R₁₅ and R₁₆ may form together with the nitrogen atom a saturated or unsaturated, optionally aromatic ring comprising from 5 to 8 total atoms, for instance, from 4 to 6 carbon atoms and/or from 2 to 4 heteroatoms chosen from O, S, and N; wherein the ring may optionally be fused with at least one saturated or unsaturated, optionally aromatic ring, each comprising from 5 to 7 total atoms, for example, from 4 to 7 carbon atoms and/or from 2 to 4 heteroatoms chosen from O, S, and N;

b) —R′₁₅—N—R′₁₆— groups wherein R′₁₅ and R′₁₆ form, together with the nitrogen atom, a saturated or unsaturated, optionally aromatic ring comprising in total from 5 to 8 atoms, for instance, from 4 to 6 carbon atoms and/or from 2 to 4 heteroatoms chosen from O, S, and N; the ring optionally being fused with at least one other saturated or unsaturated, optionally aromatic ring comprising from 5 to 7 atoms, for instance, from 4 to 8 carbon atoms and/or from 2 to 4 heteroatoms chosen from O, S, and N, for example, the ionizable group may be chosen from aromatic or non-aromatic rings comprising a cationizable tertiary amine group and aromatic or non-aromatic heterocycles containing a cationizable tertiary nitrogen;

c) guanidino and amidino groups, respectively, of formulas:

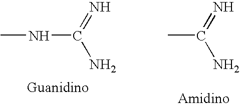

d) quaternary ammonium radicals of formula: —N⁺(R¹²)₃Z′ wherein R¹², which may be identical or different is chosen from linear or branched, C₁ to C₂₀ alkyl radicals and Z is chosen from halogen atoms such as Br and Cl, and —OSO₃CH₃;

e) and mixtures thereof.

In at least one embodiment, these radicals may be chosen from pyridyl, indolyl, isoindolinyl, imidazolyl, imidazolinyl, piperidyl, pyrazolyl, quinolinyl, pyrazolinyl, piperazinyl, pyrrolidinyl, quinidinyl, thiazolinyl, morpholinyl, guanidino, amidino radicals, and combinations thereof.

The amine units may optionally be neutralized. Non-limiting examples of salts include the salts of mineral acids, such as sulfuric acid, hydrochloric acid, and phosphoric acid. Other examples include, but are not limited to, the salts of organic acids, which may comprise at least one group chosen from carboxylic, sulfonic, and phosphonic acid groups. They may be chosen from linear, branched, or cyclic aliphatic acids and aromatic acids. These acids may also comprise at least one heteroatom chosen from O and N, for example, in the form of hydroxyl groups. Non-limiting examples include propionic acid, acetic acid, terephthalic acid, citric acid, tartaric acid, and lactic acid.

It should be noted that the neutralization of the acid or amine units, and likewise the quaternization, may be total or partial.

In at least one embodiment, the ionizable groups may be chosen from:

anionizable groups: monovalent groups —COOH, —CH₂COOH, —(CH₂)₂COOH, —(CH₂)₃COOH, —(CH₂)SO₃H, —(CH₂)₂SO₃H, —(CH₂)₃SO₃H, and —O(CH₂)₃SO₃H; and divalent groups —C(COOH)CH₃)— and —CH₂—C(COOH)(CH₃)—CH₂—;

for which the neutralizers may be chosen from NaOH, KOH, Ca(OH)₂, NH₄OH, triethylamine, butylamine, 2-amino-2-methylpropanol, triethanolamine, dimethylamino-2-propanol, lysine, and 3-(dimethylamino)propylamine;

cationizable groups: monovalent groups —(CH₂)₂—N(CH₃)₂, —N(CH₃)₂, —(CH₂)₃—N(CH₃)₂, —O—(CH₂)₃—N(CH₂CH₃)₂, and —(CH₂)₂—N(CH₂CH₃)₂; divalent groups —(CH₂)₂—N(CH₃)—(CH₂)₂— and —(CH₂)₃—N(CH₃)—(CH₂)₃—;

for which the neutralizers may be chosen from HCl, propionic acid, acetic acid, citric acid, and tartaric acid.

Examples of groups G₁ bearing ionizable groups include, but are not limited to, the groups CH₂═C(COOH)—COO— and HOC(CH₃)(COOH)—.

Thus, possible groups G₁-L-A include, but are not limited to:

the group CH₂═C(COOH)—COO—CH₂OCONH(CH₂)₆ ureidopyrimidone of formula:

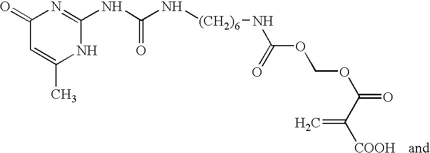

the group HOC(CH₃)(COOH)—CH₂CH₂OC(O)—NH(CH₂)₆ ureidopyrimidone of formula:

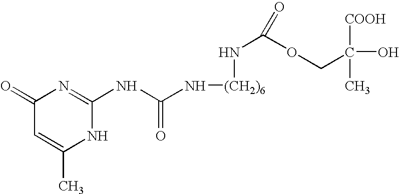

Monomers G₂ comprising a double bond that may undergo anionic, cationic, or radical polymerization and that comprises at least one ionizable group, for instance, at least one group chosen from anionizable and cationizable groups, may include, for example:

(i) ethylenically unsaturated monomers comprising at least one function chosen from carboxylic acid (COOH), phosphonic acid (PO₃H₂), and sulfonic acid (SO₃H) functions, for instance those having the following formula:
CH₂═C(R₁₉)-(Z₁)_{z1}-(Z₂)_{z2}-Y
wherein:

R₁₉ is chosen from hydrogen and linear, cyclic, or branched hydrocarbon-based radicals of the type CₚH₂ₚ₊₁, wherein p is an integer ranging from 1 to 12. In one embodiment, R₁₉ may be chosen from methyl, ethyl, propyl, and butyl radicals. In another embodiment, R₁₉ may be chosen from hydrogen, methyl radicals, and ethyl radicals;

Z₁ is a divalent group chosen from —COO—, —CONH—, —CONCH₃—, —OCO—, and —O—; and in at least one embodiment, Z₁ is chosen from —COO— and —CONH—;

z₁ is equal to 0 or 1, and in at least one embodiment, equal to 1;

Z₂ is chosen from linear, branched, or cyclic, optionally aromatic, saturated or unsaturated divalent carbon-based radicals comprising from 1 to 30 carbon atoms, which may comprise from 1 to 30 heteroatoms chosen from O, N, S, and P;

z₂ is equal to 0 or 1, and in at least one embodiment, equal to 1; and

Y is a group chosen from —COOH, —SO₃H, —OSO₃H, —PO(OH)₂, and —OPO(OH)₂.

In the radical Z₂, the at least one heteroatom, when present, may be intercalated in the chain of the radical Z₂, or alternatively the radical Z₂ may be substituted with at least one group comprising the at least one heteroatom such as hydroxyl and amino (e.g. NH₂, NHR′, and NR′R″ wherein R′ and R″, which may be identical or different, are chosen from linear or branched C₁-C₂₂ alkyl groups, such as methyl and ethyl) groups.

Z₂ may be chosen, for example from:

alkylene radicals such as methylene, ethylene, propylene, n-butylene, isobutylene, tert-butylene, n-hexylene, n-octylene, n-dodecylene, n-octadecylene, n-tetradecylene, and n-docosanylene;

phenylene radicals —C₆H₄-(ortho, meta, and para), which may be optionally substituted, with a C₁-C₁₂ alkyl radical optionally comprising from 1 to 8 heteroatoms chosen from O, N, S, and P;

benzylene radicals —C₆H₄—CH₂—, which may be optionally substituted, with a C₁-C₁₂ alkyl radical optionally comprising from 1 to 8 heteroatoms chosen from O, N, S, and P; and

radicals of formulas —CH₂—CH(OH)—, —CH₂—CH₂—CH(OH)—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, and —CH₂—CH═CH— wherein R′ and R″ are chosen from linear or branched C₁-C₁₈ alkyl radicals, such as methyl and ethyl.

Examples of anionizable monomers G₂ include, but are not limited to acrylic acid, methacrylic acid, crotonic acid, itaconic acid, fumaric acid, maleic acid, diacrylic acid, dimethylfumaric acid, citraconic acid, acrylamidopropanesulfonic acid, 2-acrylamido-2-methylpropanesulfonic acid, styrenesulfonic acid, vinylbenzoic acid, vinylphosphoric acid, vinylsulfonic acid, vinylbenzenesulfonic acid, acrylamidoglycolic acid of formula CH₂═CH—CONHCH(OH)COOH, vinylphosphonic acid, 2-carboxyethyl(meth)acrylate, sulfopropyl(meth)acrylate (CH₂═C(CH₃)CO₂(CH₂)₃SO₃H), sulfoethyl(meth)acrylate, vinyl methyl sulfone, 2-(methacryloyloxy)ethyl phosphate of formula CH₂═C(CH₃)COOC₂H₄OP(O)(OH)₂, diallyl maleate of formula C₃H₅—CO₂—CH═CH—CO₂—C₂H₅, carboxylic anhydrides bearing a vinyl bond, such as maleic anhydride, the salts thereof, and mixtures thereof.

(ii) ethylenically unsaturated monomers comprising at least one function chosen from primary, secondary, and tertiary amine functions, such as those having the following formula: 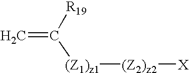
wherein:

R₁₉, Z₁, Z₂, z₁ and z₂ have the same meanings as in the above formula; and

X is (a) a group of formula —N—R₁₇R₁₈ wherein R₁₇ and R₁₈, which may be identical or different, are chosen from:

(i) hydrogen;

(ii) linear, branched, or cyclic, saturated or unsaturated, optionally aromatic alkyl groups comprising from 1 to 30 carbon atoms, which may optionally comprise from 1 to 10 heteroatoms chosen from O, N, S, and P; such as methyl, ethyl, propyl, isopropyl, n-butyl, t-butyl, isobutyl, lauryl, and stearyl groups;

(iii) alkylene oxide groups of formula —(R₂₀O)_{y}R₂₁ wherein R₂₀ is chosen from linear or branched C₂-C₄ alkyl radicals, R₂₁ is chosen from hydrogen and linear or branched C₂-C₃₀ alkyl radicals, and y is a number ranging from 1 to 250;

(iv) R₁₇ and R₁₈ may form, together with the nitrogen atom, a saturated or unsaturated optionally aromatic ring comprising in total from 5 to 8 atoms, for instance, from 4 to 6 carbon atoms and/or from 2 to 4 heteroatoms chosen from O, S, and N; the ring optionally being fused with at least one other saturated or unsaturated, optionally aromatic ring comprising from 5 to 7 atoms, for example, from 4 to 7 carbon atoms and/or from 2 to 4 heteroatoms chosen from O, S, and N;

or alternatively X is (b) a group —R′₁₅—N—R′₁₆— wherein R′₁₅ and R′₁₆ form, together with the nitrogen atom, a saturated or unsaturated, optionally aromatic ring, comprising in total from 5 to 8 atoms, for example, from 4 to 6 carbon atoms and/or from 2 to 4 heteroatoms chosen from O, S, and N; the ring optionally being fused with at least one saturated or unsaturated, optionally aromatic ring comprising from 5 to 7 atoms, for instance, from 4 to 8 carbon atoms and/or from 2 to 4 heteroatoms chosen from O, S, and N.

For example, X may be chosen from aromatic or non-aromatic rings comprising a cationizable tertiary amine group and aromatic or non-aromatic heterocycles comprising a cationizable tertiary nitrogen. Non-limiting examples of radicals X include pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino, and amidino radicals, and mixtures thereof.

According to one embodiment, the cationizable monomers G₂ may be chosen from:

2-vinylpyridine, 4-vinylpyridine, allylamine, and allylpyridine;

aminoalkyl(meth)acrylates, such as [N,N-di(C₁-C₄)alkylamino](C₁-C₆)alkyl(meth)acrylates and [N—(C₁-C₄)alkylamino](C₁-C₆)alkyl(meth)acrylates, for instance, N,N-dimethylaminoethyl(meth)acrylate, N,N-diethylaminoethyl(meth)acrylate, 2-aminoethyl(meth)acrylate, and 2-(N-tert-butylamino)ethyl(meth)acrylate;

aminoalkyl(meth)acrylamides, such as [N,N-di(C₁-C₄)alkylamino](C₁-C₆)alkyl(meth)acrylamides and [N—(C₁-C₄)alkylamino](C₁-C₆)alkyl(meth)acrylamides, for example, N,N-dimethylaminopropyl(meth)acrylamide, N,N-dimethylaminoethyl(meth)acrylamide and 3-aminopropyl(meth)acrylamide;

vinylamine, vinylimidazole, and 2-(diethylamino)ethylstyrene;

N-vinylimidazole, N-vinyl-2-methylimidazole, and N-vinylcarbazole;

the salts thereof;

the quaternized forms thereof; and

mixtures thereof.

(iii) monomers of formula: 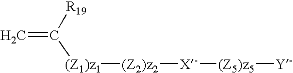
wherein:

R₁₉, Z₁, Z₂, z₁, and z₂ have the same meaning as in the above formula,

Z₅ has the same meaning as that given for Z₂, but may be different from Z₂,

z₅=0 or 1,

X′⁺ is a divalent group of formula —N⁺(R₆)(R₇)— wherein R₆ and R₇, which may be identical or different, are chosen from (i) a hydrogen, (ii) a linear, branched, or cyclic, optionally aromatic alkyl groups comprising from 1 to 25 carbon atoms, which may optionally comprise from 1 to 20 heteroatoms chosen from O, N, S, and P; and (iii) R₆ and R₇ may form, together with the nitrogen atom a first saturated or unsaturated, optionally aromatic ring comprising in total from 5 to 8 atoms, for example, from 4 to 7 carbon atoms and/or from 2 to 3 heteroatoms chosen from O, N, and S; the first ring possibly being fused with at least one other saturated or unsaturated, optionally aromatic rings, each comprising from 5 to 8 atoms, for example, from 4 to 7 carbon atoms and/or from 2 to 3 heteroatoms chosen from O, N, and S; and in at least one embodiment, R₆ and R₇, are chosen from hydrogen, methyl, ethyl, propyl, isopropyl, n-butyl, t-butyl, and isobutyl groups; and

Y′⁻ is a group chosen from —COO⁻, —SO₃⁻, —OSO₃⁻, —PO₃²⁻, and OPO₃²⁻.

According to one embodiment, Z₂ and Z₅ may be chosen from saturated or unsaturated, linear, branched, or cyclic (aromatic or non-aromatic) alkylene groups comprising from 1 to 30 carbon atoms, optionally including at least one heteroatom such as O, N, S, and P. In another embodiment, Z₂ and Z₅ may be chosen from C₁-C₃₀ alkylene, phenylene, benzylene, —(CH₂—CH═CH)—, —(CH₂—CH₂—CHOH)—, and —CH₂—CH₂—CH(NR₂₀R₂₁)— groups wherein R₂₀ and R₂₁, which may be identical or different, are chosen from H and C₁-C₁₈ alkyls, for example, R₂₀ and R₂₁ may be chosen from H, CH₃, and C₂H₅. According to a further embodiment, Z₂ and Z₅ may be chosen from C₁-C₆ alkylene, phenylene, and benzylene groups.

Mention may also be made of N,N-dimethyl-N-(2-methacryloyloxyethyl)-N-(3-sulfopropyl)ammonium betaine (for example, SPE from the company Raschig); N,N-dimethyl-N-(3-methacrylamidopropyl)-N-(3-sulfopropyl)ammonium betaine (SPP from Raschig) and 1-(3-sulfopropyl)-2-vinylpyridinium betaine (SPV from Raschig).

The monomers G₂ that can (co)polymerize via polyaddition or polycondensation and comprise at least one ionizable group, may include the monomers of formula HX″—B—X″H, wherein:

X″, which may be identical or different, is chosen from O, S, NH, and NR, wherein R is chosen from C₁₋₆ alkyl groups,

B is chosen from linear, branched, or cyclic, saturated or unsaturated, optionally aromatic alkylene radicals comprising from 1 to 6000 carbon atoms, optionally comprising at least one heteroatom chosen from O, S, P, and N, and/or optionally substituted with at least one atom chosen from fluorine and silicon atoms;

with the proviso that at least one of the radicals B bears an ionizable group as defined above.

According to one embodiment, B may be chosen from:

alkylene radicals comprising from 1 to 40 carbon atoms and cycloalkylene radicals comprising from 3 to 16 carbon atoms, optionally substituted with a C₁-C₁₂ alkyl radical and/or optionally comprising from 1 to 8 heteroatoms chosen from O, N, S, F, Si, and P; such as methylene, ethylene, propylene, n-butylene, isobutylene, tert-butylene, n-hexylene, n-octylene, n-dodecylene, n-octadecylene, n-tetradecylene, n-docosanylene, 2-ethylhexyl, cyclohexylene, cyclohexylmethylene, and isophorone;

C₁-C₃₀ arylene radicals such as a phenylene radical —C₆H₄-(ortho, meta, or para);

C₁ to C₃₀, for instance, C₂ to C₁₂, alkylarylene and arylalkylene radicals optionally substituted with a C₁-C₁₂ alkyl radical optionally comprising from 1 to 25 heteroatoms chosen from O, N, S, F, Si, and P; such as a benzylene radical —C₆H₄—CH₂— optionally substituted with a C₁-C₁₂ alkyl radical optionally comprising from 1 to 8 heteroatoms chosen from O, N, S, F, Si, and P.

When present in B, the at least one heteroatom may be intercalated in the chain of the radical, or alternatively the radical may be substituted with at least one group comprising the at least one heteroatom, such as hydroxyl and amino (NH₂, NHR′, and NR′R″ wherein R′ and R″, which may be identical or different, are chosen from linear or branched C₁-C₂₂ alkyl radicals optionally comprising from 1 to 12 heteroatoms chosen from O, N, S, F, Si, and P, for example, methyl or ethyl).

In one embodiment, B may comprise at least one radical chosen from;

radicals of formulas —CO—O—, —CO—O—, —OCO—, —O—CO—NH—, anhydride, —NH—CO—NH—, and NHCO;

radicals —Si(R₄)(R₅)O— wherein R₄ and R₅, which may be identical or different, are chosen from H and linear or branched, cyclic or non-cyclic, saturated or unsaturated, or aromatic hydrocarbon-based radicals, for example, C₁-C₁₂ alkyl radicals optionally comprising at least one, for instance, from 1 to 5, identical or different heteroatom chosen from O, N, S, P, F, and Si, and in at least one embodiment, O, N, and S; and

oxyalkylene and aminoalkylene radicals, for example, alkylene oxide radicals of formula —(R″O)_{y}R″₁ wherein R″ is chosen from linear or branched C₂-C₄ alkyl radicals, R″₁ is chosen from H and linear or branched C₁ to C₃₀ alkyl radicals, and y is a number ranging from 1 to 500.

In at least one embodiment, the monomers HX—B—XH that can (co)polymerize via polyaddition or polycondensation, and that comprise at least one ionizable group may be chosen from dimethylolpropionic acid, dimethylaminopropionic acid, N-ethylsulfonicdimethanolamine, N-ethylsulfonicdiethanolamine, benzenesulfonic acid diol, diaminopyridine, N-methyldiethanolamine, N-ethyldiethanolamine, N-tert-butyl-diethanolamine, and mixtures thereof.

It has been found that the (co)polymers according to the present disclosure may be advantageously soluble or dispersible in aqueous media, carbon-based oils, silicone oils, and/or cosmetic solvents, such as alkanols, for instance, ethanol, and alkyl esters such as alkyl acetates.

According to at least one embodiment, the (co)polymers of the present disclosure may be soluble or dispersible in water.

The (co)polymer is said to be soluble in the medium when it is dissolved, i.e., when it forms a clear solution, at a proportion of at least 1% by weight in the medium at 25° C.

The (co)polymer is said to be dispersible if it forms in the medium, at a concentration of 1% by weight at 25° C., a stable suspension or dispersion of fine, generally spherical particles. As used herein, the term “stable” means that the suspension does not precipitate and therefore does not have a visible deposit. The mean size of the particles constituting the suspension or dispersion may be less than 1 μm, for example, ranging from 5 to 400 nm, or from 10 to 250 nm. These particle sizes may be measured by any standard light scattering method.

The (co)polymers according to the present disclosure may be chosen from compounds belonging to one of the following categories, with the proviso that they comprise at least one ionizable group:

(i) ethylenic, such as vinyl (co)polymers, for instance (meth)acrylic copolymers, (meth)acrylamide copolymers, allylic copolymers, copolyolefins such as hydrogenated or non-hydrogenated polydienes, and mixtures thereof.

For example, the (co)polymers according to the present disclosure may be chosen from vinyl/(meth)acrylate, vinyl/(meth)acrylamide, vinyl/(meth)acrylate/methacrylamide, olefinic/vinyl, and (meth)acrylate/(meth)acrylamide copolymers.

Additional examples of these copolymers include, but are not limited to, copolymers based on vinyl acetate, styrene, vinylpyrrolidone, vinylcaprolactam, polyethylene oxide(meth)acrylate, stearyl(meth)acrylate, lauryl(meth)acrylate, vinyllaurate, butyl(meth)acrylate, ethylhexyl(meth)acrylate, crotonic acid, (meth)acrylic acid, maleic anhydride, styrenesulfonic acid, dimethyldiallylamine, vinylpyridine, dimethylaminoethyl(meth)acrylate, dimethylaminopropyl(meth)acrylamide, and salts thereof.

In this embodiment, the (co)polymers obtained may comprise junction units (A) as grafts along the backbone of the (co)polymer;

(ii) polycondensates of polyurethane and/or polyureas, aliphatic or aromatic polyesters, aliphatic or aromatic polyamides, and copolymers thereof, for instance, polyurethane/urea, polyester/amide, and polyester/polyurethane/urea;

(iii) (co)polymers obtained via ring opening, for instance, polyethers such as polyethylene oxide, polypropylene oxide, and copolymers thereof polyethylene oxide/polypropylene oxide; polylactides, polyesters, for instance, polycaprolactone; polyoxazolines such as poly(2-methyloxazoline) and poly(2-ethyloxazoline);

(iv) siloxane (co)polymers, for instance, polydimethylsiloxanes (PDMS) and polymethylphenylsiloxanes;

(v) polythioethers, polycarbonates, polyacetals, and perfluoropolyethers;

(vi) (co)polymers obtained via metathesis, for instance, poly(norbornene) and copolymers thereof;

(vii) copolymers of various types of polymer, for instance, polysiloxane/polyethylene oxide, polysiloxane/polyurethane/urea, poly(ethylene-butylene)/polyurethane, and hydrogenated polybutadiene/polyurethane copolymers;

(viii) salts thereof; derivatives thereof; and mixtures thereof.

The (co)polymers according to the present disclosure may be useful in the field of cosmetics. They may be present in the composition in dissolved form, for example, in a solvent medium that may comprise water and/or at least one organic solvent, or alternatively in the form of an aqueous or organic dispersion.

The (co)polymers may be used in compositions, for example, cosmetic and pharmaceutical compositions, in an amount ranging from 0.01% to 90% by weight of solids, for example, from 0.05% to 70% by weight, from 0.1% to 60% by weight, or from 1% to 50% by weight, relative to the total weight of the composition.

According to at least one embodiment, the compositions according to the present disclosure may comprise a mixture of (co)polymers.

The (co)polymers POL-(A)i of a mixture of (co)polymers may comprise in pairs a different number of junction groups (A) and junction groups (A) of different nature, it being understood that the pairing condition is satisfied.

In one embodiment, the compositions according to the present disclosure may comprise at least one (co)polymer chosen from those described in International Patent Application Publication No. WO 02/98377, i.e., at least one linear, branched or cyclic polymer, or dendrimer, comprising a polymer backbone -POL- comprising at least two repeating units, and at least two junction groups (A) attached to the polymer backbone, and capable of establishing at least three H bonds with the same partner junction group. According to another embodiment, these (co)polymers may be present in the composition in an amount ranging from 0.01% to 90% by weight of solids, for example, from 0.05% to 70% by weight, from 0.1% to 60% by weight, or from 1% to 50% by weight, relative to the total weight of the composition

The cosmetic and/or pharmaceutical compositions according to the present disclosure may comprise, besides the (co)polymers, a physiologically acceptable medium, for instance, a cosmetically or dermatologically acceptable medium, i.e., a medium that is compatible with keratin materials such as facial and/or bodily skin, the hair, the eyelashes, the eyebrows, and the nails.

The physiologically acceptable medium is, in at least one embodiment, a medium that does not harm the properties of increased persistence of at least one cosmetic and/or care effect, of adhesion to keratin materials, and of ease of makeup removal provided by the composition after application.

The physiologically acceptable medium may comprises a solvent medium for the (co)polymers according to the present disclosure, which may comprise at least one compound chosen from water, alcohols, polyols, esters, carbon-based oils, silicone oils, fluorosilicone oils, and mixtures thereof.

Thus, the solvent medium for the compositions according to the present disclosure may be chosen from water and mixtures of water and of at least one hydrophilic organic solvent, for instance, alcohols such as linear or branched C₁-C₆ monoalcohols, for instance, ethanol, tert-butanol, n-butanol, isopropanol, n-propanol, and 2-butoxyethanol; and polyols, for instance, glycerol, diglycerol, ethylene glycol, propylene glycol, sorbitol, pentylene glycol, polyethylene glycols, and glycol ethers, for example, those of C₂, such as diethylene glycol monoethyl ether and monomethyl ether, and hydrophilic C₂-C₄ aldehydes.

The solvent medium may comprise aromatic alcohols, for instance, benzyl alcohol and phenoxyethanol and derivatives thereof; carboxylic esters, and mixtures thereof.

Examples of carboxylic esters include, but are not limited to, those comprising from 2 to 8 carbon atoms, such as ethyl acetate, butyl acetate, methyl acetate, propyl acetate, n-butyl acetate, and isopentyl acetate.

The solvent medium may also comprise physiologically acceptable organic solvents chosen from ketones that are liquid at room temperature, such as methyl ethyl ketone, methyl isobutyl ketone, diisobutyl ketone, isophorone, cyclohexanone, and acetone; propylene glycol ethers that are liquid at room temperature, such as propylene glycol monomethyl ether, propylene glycol monomethyl ether acetate, and dipropylene glycol mono-n-butyl ether; ethers that are liquid at 25° C., such as diethyl ether, dimethyl ether, and dichlorodiethyl ether; alkanes that are liquid at 25° C., such as decane, heptane, dodecane, isododecane, and cyclohexane; cyclic aromatic compounds that are liquid at 25° C., such as toluene and xylene; aldehydes that are liquid at 25° C., such as benzaldehyde and acetaldehyde; and mixtures thereof.

The solvent medium may also comprise polar or apolar, carbon-based or silicone-based cosmetic oils of animal, plant, mineral, or synthetic origin.

Suitable polar oils may include, for example, carbon-based oils that may comprise ester, ether, acid, and/or alcohol functions, for instance:

carbon-based plant oils with a high triglyceride content comprising fatty acid esters of glycerol, the fatty acids of which may have varied chain lengths, these chains possibly being linear or branched, and saturated or unsaturated; for example, wheatgerm oil, corn oil, sunflower oil, shea oil, castor oil, sweet almond oil, macadamia oil, apricot oil, soybean oil, rapeseed oil, cottonseed oil, alfalfa oil, poppy oil, pumpkin oil, sesameseed oil, marrow oil, avocado oil, hazelnut oil, grapeseed oil, blackcurrant pip oil, evening primrose oil, millet oil, barley oil, quinoa oil, olive oil, rye oil, safflower oil, candlenut oil, passion flower oil, and musk rose oil; and caprylic/capric acid triglyercides, for instance, those sold by the company Stearineries Dubois and those sold under the names Miglyol 810, 812, and 818 by the company Dynamit Nobel;

synthetic oils of formula R²⁰COOR²¹ wherein R²⁰ is chosen from linear or branched fatty acid residues comprising from 7 to 19 carbon atoms and R²¹ is chosen from branched hydrocarbon-based chains comprising from 3 to 20 carbon atoms, for instance Purcellin oil (cetostearyl octanoate), isononyl isononanoate, and C₁₂-C₁₅ alkyl benzoates;

synthetic esters and ethers, for instance, isopropyl myristate, 2-ethylhexyl palmitate and alcohol or polyalcohol octanoates, decanoates, and ricinoleates;

hydroxylated esters, for instance, isostearyl lactate, diisostearyl malate, and pentaerythritol esters;

C₈ to C₂₆ fatty alcohols, for instance, oleyl alcohol; and

mixtures thereof.

Non-limiting examples of apolar oils include:

volatile or non-volatile, linear or cyclic silicone oils that are liquid at room temperature, such as polydimethylsiloxanes (PDMS) comprising at least one group chosen from alkyl, alkoxy, and phenyl groups, which are pendent and/or at the end of a silicone chain and which comprise from 2 to 24 carbon atoms; phenyl silicones, for instance, phenyl trimethicones, phenyl dimethicones, phenyl trimethylsiloxydiphenylsiloxanes, diphenyl dimethicones, diphenylmethyltrisiloxanes, and 2-phenylethyl trimethylsiloxysilicates,

linear or branched hydrocarbons, fluorohydrocarbons, and fluorocarbons of synthetic or mineral origin, for instance, volatile oils, such as liquid paraffins (for example, isoparaffins such as isododecane), and non-volatile oils and derivatives thereof, such as petroleum jelly, polydecenes, hydrogenated polyisobutene such as parleam, squalane, and mixtures thereof.

The solvent medium may preferably be present in the composition in an amount ranging from 1% to 90% by weight, for example, from 5% to 70% by weight relative to the total weight of the composition.

The solubility of the (co)polymers according to the present disclosure may be controlled by the choice of polymer backbones -POL- and/or junction groups (A).

The (co)polymers of the present disclosure may in certain cases interact physically with each other (by establishing a network of H interactions) in certain solvents or solvent mixtures. This may depend on the nature and proportions of solvents or solvent mixtures used. This may cause an undesirable increase in the viscosity of the composition and possibly interfere with its application (for example, lotion, aerosol, etc.).

To overcome this potential viscosity problem, it is possible, in at least one embodiment:

to dissolve the (co)polymer according to the present disclosure in a volatile solvent capable of establishing H interactions with junction groups (A), for example, by using C₁-C₄ short alcohols, volatile polyols, water, and/or a mixture of these solvents, or

to use a two-phase solvent medium, for instance, a water-in-oil (W/O) or oil-in-water (O/W) emulsion and a pair of selective (co)polymers according to the present disclosure whose junction groups and polymer backbones are of different chemical nature, each polymer being dissolved in a different phase from the other (one in water, the other in the oil).

In the latter case, the pair of (co)polymers A₁-POL₁-A₁ and A₂-POL₂-A₂ may be chosen such that:

each of the junction groups (A₁) does not establish H interactions with itself but only with (A₂),

each of the junction groups (A₂) does not establish H interactions with itself but only with (A₁),

the junction groups (A₁) and (A₂) establish H interactions only when they are placed in contact, and

the polymer backbones -POL₁- and -POL₂- are chosen such that the (co)polymers A₁-POL₁-A₁ and A₂-POL₂-A₂ may each be conveyed in a different phase of the emulsion, such that they cannot react together in the emulsion.

According to these embodiments, the interaction between the two (co)polymers will take place only on application, provided, however, that the solvent media are volatile solvents or solvents that can penetrate into the keratin support.

The composition of the present disclosure may also comprise at least one adjuvant commonly used in cosmetics and/or pharmaceuticals, provided that the adjuvant does not impair the desired properties for the composition of the present disclosure, such as waxes, gums, surfactants, thickeners, hydrophilic or lipophilic gelling agents, hydrophilic or lipophilic cosmetic active agents, preserving agents, antioxidants, fragrances, nacreous agents, fillers, neutralizers, (co)polymers other than those defined above, emulsifiers and co-emulsifiers, pigments, and dyestuffs.

It is to be understood that a person skilled in the art will take care to select the at least one optional adjuvant and/or the amount thereof such that the advantageous properties of the composition according to the present disclosure are not, or are not substantially, adversely affected by the envisaged addition.

The at least one adjuvant may be present in the composition in amounts conventionally used in the fields under consideration, for example, from 0.001% to 30% by weight relative to the total weight of the composition. Depending on its nature, the at least one adjuvant may be introduced into the fatty phase, into the aqueous phase, into lipid vesicles, and/or into nanoparticles.

As used herein, the term “wax” means a lipophilic compound that is solid at room temperature (25° C.), which undergoes a reversible solid/liquid change of state, and which has a melting point of greater than or equal to 25° C., which may be up to 120° C. By bringing the wax to the liquid state (melting), it is possible to make it miscible with the oils possibly present and to form a microscopically homogeneous mixture, but, on returning the temperature of the mixture to room temperature, recrystallization of the wax is obtained in the oils of the mixture. The melting point of the wax may be measured using a differential scanning calorimeter (DSC), for example, the calorimeter sold under the name DSC 30 by the company Mettler.

The waxes may be chosen from hydrocarbon-based waxes, fluoro waxes, and/or silicone waxes and may be of plant, mineral, animal, and/or synthetic origin. In at least one embodiment, the waxes may have a melting point of greater than 30° C., for example, greater than 45° C. Examples of waxes that may be used in the composition of the present disclosure include, but are not limited to, beeswax, carnauba wax, candelilla wax, paraffin, microcrystalline waxes, ceresin, ozokerite; synthetic waxes, for instance, polyethylene waxes and Fischer-Tropsch waxes, and silicone waxes, for instance, alkyl and alkoxy dimethicones comprising from 16 to 45 carbon atoms.

The gums generally may be chosen from polydimethylsiloxanes (PDMSs) of high molecular weight, cellulose gums, and polysaccharides, and the pasty substances generally may be hydrocarbon-based compounds, for instance, lanolins and derivatives thereof, and PDMSs.

The nature and amount of the waxes and/or gums depend on the desired mechanical properties and textures of the composition. As a general guide, the at least one wax may be present in the composition in an amount ranging from 0.1% to 50% by weight, or from 1% to 30% by weight, relative to the total weight of the composition.

The composition according to the invention may also comprise, in a particulate phase, at least one pigment and/or at least one nacre, and/or at least one filler conventionally used in cosmetic compositions.

The composition may also comprise other dyestuffs chosen from water-soluble dyes and/or liposoluble dyes that are known in the art.

As used herein, the term “pigments” should be understood as meaning white or colored, mineral or organic particles of any shape, which are insoluble in the physiological medium and which are intended to color the composition.

As used herein, the term “fillers” should be understood as meaning colorless or white, mineral or synthetic, lamellar or non-lamellar particles intended to give body or rigidity to the composition, softness, a matt effect, and/or uniformity to the makeup.

As used herein, the term “nacres” should be understood as meaning iridescent particles of any shape, produced, for instance, by certain molluscs in their shell, or else synthesized.

The at least one pigment may be present in the composition in an amount ranging from 0.01% to 25%, for example, from 3% to 10% by weight of the final composition. They may be white or colored, and mineral or organic. Examples include, but are not limited to, titanium oxide, zirconium oxide, cerium oxide, zinc oxide, iron oxide, chromium oxide, ferric blue, chromium hydrate, carbon black, ultramarines (aluminosilicate polysulfides), manganese pyrophosphate, and certain metallic powders such as silver and aluminium powders. The pigments may also be chosen from the D&C pigments and lakes commonly used to give the lips and the skin a makeup effect, which include, for example, calcium, barium, aluminium, strontium, and zirconium salts.

The at least one nacre may be present in the composition in an amount ranging from 0.01% to 20% by weight, for example, from 3% to 10% by weight. Non-limiting examples of nacres include natural mother-of-pearl, mica coated with titanium oxide, mica coated with iron oxide, mica coated with natural pigment, mica coated with bismuth oxychloride, and colored titanium mica.

Examples of liposoluble or water-soluble dyes that may be present in the composition, alone or as a mixture, in an amount ranging from 0.001% to 15% by weight, for instance, from 0.01% to 5% by weight, or from 0.1% to 2% by weight, relative to the total weight of the composition, include, but are not limited to, the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red, annatto, beetroot juice, and carotene.

The composition according to the present disclosure may also comprise at least one filler, present in an amount ranging from 0.01% to 50% by weight, for example, from 0.02% to 30% by weight, relative to the total weight of the composition. The fillers may be mineral or organic in any shape, such as platelet-shaped, spherical, and oblong. Suitable fillers include, but are not limited to, talc, mica, silica, kaolin, polyamide (Nylon®) powder, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile (for instance, Expancel® (Nobel Industrie) and those of acrylic acid copolymers (for example, Polytrap® from the company Dow Corning), silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, for instance, from 12 to 18 carbon atoms, for example zinc, magnesium stearate, lithium stearate, zinc laurate, and magnesium myristate.

The composition may also comprise at least one additional polymer such as a film-forming polymer. According to the present disclosure, the term “film-forming polymer” means a polymer capable, by itself or in the presence of an auxiliary film-forming agent, of forming a continuous film that adheres to a support such as keratin materials. Examples of film-forming polymers include, but are not limited to, synthetic polymers, free-radical polymers, polycondensate polymers, polymers of natural origin, and mixtures thereof, for instance, acrylic polymers, polyurethanes, polyesters, polyamides, polyureas, and cellulose-based polymers, for example, nitrocellulose.

The composition may also comprise at least one surfactant, which may be present in an amount ranging from 0.01% to 50% by weight, for example, from 0.1% to 40%, or from 0.5% to 30%, relative to the total weight of the composition.

This at least one surfactant may be chosen from anionic, amphoteric, nonionic, and cationic surfactants, and mixtures thereof.

Surfactants that are suitable for use in accordance with the present disclosure may include, for example, the following surfactants and mixtures thereof:

anionic surfactants, for example, salts (such as alkali metal salts, for instance, sodium salts, ammonium salts, amine salts, amino alcohol salts, and magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates, and N-acyltaurates; and mixtures thereof; wherein the alkyl or acyl radical of these various compounds comprises, for example, from 8 to 24 carbon atoms, and the aryl radical is chosen from phenyl and benzyl groups.

Other non-limiting examples include fatty acid salts such as the salts of oleic, ricinoleic, palmitic and stearic acids, coconut oil acid, and hydrogenated coconut oil acid; acyl lactylates in which the acyl radical comprise from 8 to 20 carbon atoms; alkyl-D-galactosiduronic acids and their salts, polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for instance, those comprising from 2 to 50 ethylene oxide groups, and mixtures thereof;

nonionic surfactants, for example, polyethoxylated fatty acid, polypropoxylated fatty acids, polyglycerolated fatty acids, alkylphenols, α-diols and alcohols comprising a fatty chain comprising, for example, from 8 to 18 carbon atoms, and mixtures thereof; it being possible for the number of ethylene oxide or propylene oxide groups to range, for example, from 2 to 50 and for the number of glycerol groups to range, for instance, from 2 to 30.

Further non-limiting examples include copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average from 1 to 5, for instance, from 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides, and N-acylaminopropylmorpholine oxides;

amphoteric surfactants, for example, aliphatic secondary or tertiary amine derivatives in which the aliphatic radical is a linear or branched chain comprising from 8 to 22 carbon atoms and comprising at least one water-soluble anionic group (for example, carboxylate, sulfonate, sulfate, phosphate, and phosphonate); (C₈-C₂₀)alkylbetaines, sulfobetaines, (C₈-C₂₀)alkylamido(C₁-C₆)alkylbetaines such as cocoamidopropylbetaine, (C₈-C₂₀)alkylamido(C₁-C₆)alkylsulfobetaines, and mixture thereof;

cationic surfactants, such as the following surfactants and mixtures thereof:

A) quaternary ammonium salts of formula (XVI): 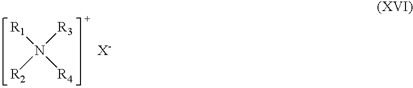
wherein X is an anion chosen from halides (e.g., chloride, bromide, and iodide) and (C₂-C₆)alkyl sulfates, such as methyl sulfate, phosphates, alkyl and alkylaryl sulfonates, and anions derived from organic acid, such as acetate and lactate, and

a) the radicals R₁, R₂, and R₃, which may be identical or different, are chosen from linear or branched aliphatic radicals comprising from 1 to 4 carbon atoms and aromatic radicals such as aryl and alkylaryl radicals. The aliphatic radicals may optionally comprise at least one heteroatom, such as oxygen, nitrogen, sulfur, and halogens. The aliphatic radicals may be chosen, for example, from alkyl, alkoxy, and alkylamide radicals, and

R₄ is chosen from linear or branched alkyl radicals comprising from 16 to 30 carbon atoms.

In at least one embodiment, the cationic surfactant is chosen from behenyltrimethylammonium salts (for example, behenyltrimethylammonium chloride).

b) the radicals R₁ and R₂, which may be identical or different, are chosen from linear or branched aliphatic radicals comprising from 1 to 4 carbon atoms and aromatic radicals such as aryl and alkylaryl. The aliphatic radicals may optionally comprise at least heteroatom, such as oxygen, nitrogen, sulfur, and halogens. The aliphatic radicals may be chosen, for example, from alkyl, alkoxy, alkylamide, and hydroxyalkyl radicals comprising from 1 to 4 carbon atoms; and

R₃ and R₄, which may be identical or different, are chosen from linear or branched alkyl radicals comprising from 12 to 30 carbon atoms and at least one function chosen from ester and amide functions.

In at least one embodiment, R₃ and R₄ may be chosen from (C₁₂-C₂₂)alkylamido(C₂-C₆)alkyl and (C₁₂-C₂₂)alkylacetate radicals;

According to another embodiment, the cationic surfactant may be chosen from stearamidopropyldimethyl(myristyl acetate)ammonium salts (for example, stearamidopropyldimethyl(myristyl acetate)ammonium chloride).

B)—quaternary ammonium salts of imidazolinium, for example, those of formula (XVII): 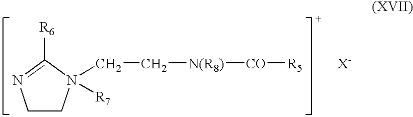
wherein:

R₅ is chosen from alkenyl and alkyl radicals comprising from 8 to 30 carbon atoms, for example fatty acid derivatives of tallow,

R₆ is chosen from hydrogen, C₁-C₄ alkyl radicals, and alkenyl and alkyl radicals comprising from 8 to 30 carbon atoms,

R₇ is chosen from C₁-C₄ alkyl radicals,

R₈ is chosen from hydrogen and C₁-C₄ alkyl radicals, and

X is an anion chosen from halides, phosphates, acetates, lactates, alkyl sulfates, alkyl sulfonates, and alkylaryl sulfonates.

In at least one embodiment, R₅ and R₆ may be chosen from mixtures of alkenyl and alkyl radicals comprising from 12 to 21 carbon atoms, for example, fatty acid derivatives of tallow, R₇ is methyl, and R₈ is hydrogen. Such a product is commercially available, for example, as Quaternium-27 (CTFA 1997) and Quaternium-83 (CTFA 1997), which are sold under the names “Rewoquat” W75, W90, W75PG, and W75HPG by the company Witco,

C)—diquaternary ammonium salts of formula (XVIII): 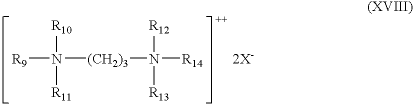
wherein:

R₉ is chosen from aliphatic radicals comprising from 16 to 30 carbon atoms,

R₁₀, R₁₁, R₁₂, R₁₃, and R₁₄, which may be identical or different, are chosen from hydrogen and alkyl radicals comprising from 1 to 4 carbon atoms, and

X is an anion chosen from halides, acetates, phosphates, nitrates, and methyl sulfates. Such diquaternary ammonium salts include, for example, propanetallowediammonium dichloride;

D)—quaternary ammonium salts comprising at least one ester function, of formula (XIX): 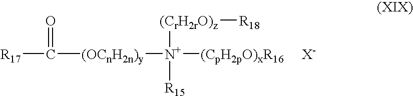
wherein.

R₁₅ is chosen from C₁-C₆ alkyl radicals, C₁-C₆ hydroxyalkyl radicals, and C₁-C₆ dihydroxyalkyl radicals;

R₁₆ is chosen from hydrogen, R₁₉—C(O)— radicals, and linear or branched, saturated or unsaturated C₁-C₂₂ hydrocarbon-based radicals R₂₀,

R₁₈ is chosen from hydrogen, R₂₁—C(O)— radicals, and linear or branched, saturated or unsaturated C₁-C₂₂ hydrocarbon-based radicals R₂₂;

R₁₇, R₁₉, and R₂₁, which may be identical or different, are chosen from linear or branched, saturated or unsaturated C₇-C₂₂ hydrocarbon-based radicals;

n, p, and r, which may be identical or different, are integers ranging from 2 to 6;

y is an integer ranging from 1 to 10;

x and z, which may be identical or different, are integers ranging from 0 to 10; and

X⁻ is a simple or complex, organic or inorganic anion; with the provisos that the sum x+y+z ranges from 1 to 15, that when x is 0, then R₁₆ denotes R₂₀, and that when z is 0, then R₁₈ denotes R₂₂.

The composition may also comprise at least one emulsifying agent chosen from emulsifiers and co-emulsifiers, for example, in emulsion form, such as, depending on the nature of the emulsion (e.g., W/O and O/W), fatty acid esters of polyols such as PEG-100 stearate, PEG-50 stearate, and PEG-40 stearate; sorbitan tristearate, oxyethylenated sorbitan stearates comprising, for example, from 20 to 100 EO, for example those available under the trade names Tween® 20 and Tween® 60 and, mixtures thereof such as the mixture of glyceryl monostearate and of polyethylene glycol stearate (100 EO) sold under the name Simulsol 165 by the company SEPPIC.

Further examples include, but are not limited to, silicone emulsifiers such as dimethicone copolyols and alkyl dimethicone copolyols. A non-limiting example of a dimethicone copolyol is the mixture of dimethicone copolyol, cyclomethicone, and water (10/88/2) sold by the company Dow Corning under the names DC3225C and DC2-5225C, and non-limiting examples of alkyl dimethicone copolyols include those with an alkyl radical comprising from 10 to 22 carbon atoms, such as cetyl dimethicone copolyol, for instance, the product sold under the name Abil EM-90 by the company Goldschmidt and the mixture of dimethicone copolyol and of cyclopentasiloxane (85/15) sold under the name Abil EM-97 by the company Goldschmidt; lauryl dimethicone copolyol, for example, the mixture of about 91% lauryl dimethicone copolyol and of about 9% isostearyl alcohol, sold under the name Q2-5200 by the company Dow Corning, and mixtures thereof.

These at least one emulsifying agent chosen from emulsifiers and co-emulsifiers may be present in the composition in an amount ranging from 0.3% to 30% by weight, for example, from 0.5% to 20% by weight relative to the total weight of the composition.

Hydrophilic gelling agents suitable for use in the compositions of the present disclosure include, for instance, carboxyvinyl polymers (carbomer), acrylic copolymers such as acrylate/alkylacrylate copolymers, polyacrylamides, polysaccharides, natural gums, and clays.

Examples of lipophilic gelling agents include, but are not limited to, modified clays, for instance bentones, metal salts of fatty acids, and hydrophobic silica.

Cosmetic active agents that may be used include, for instance, depigmenting agents, emollients, moisturizers, trace elements, anti-seborrhoeic agents, antiacne agents, hair restorers, keratolytic and/or desquamating agents, anti-wrinkle agents, tensioning agents, anti-irritant agents, calmatives, vitamins, UV-screening agents, odor absorbers, antioxidants, hair-loss counteractants, antidandruff agents, propellants, ceramides, and mixtures thereof.

The compositions according to the present disclosure may be in any galenical form conventionally used for topical application, for example, aqueous, alcoholic, and aqueous-alcoholic solutions, dispersions and suspensions and oily solutions, which may be optionally thickened or gelled; oil-in-water, water-in-oil, and multiple emulsions having a consistency chosen from liquid or semi-liquid consistencies of the milk type and soft consistencies of cream type; aqueous gels and anhydrous gels, mousses; oily or emulsified gels; dispersions of vesicles, such as lipid vesicles; two-phase lotions and multiphase lotions; sprays; and any other cosmetic form.

It is to be understood that a person skilled in the art may select the appropriate galenical form and the method for preparing it on the basis of his general knowledge, taking into account the nature of the constituents used, for example, their solubility in the support, and the intended use of the composition.

The cosmetic composition according to the present disclosure may be in a form chosen from products for caring for, cleansing, and/or making up bodily skin, facial skin, the lips and/or the hair, antisun and/or self-tanning products, body hygiene products, and hair products, such as those for caring for, cleansing, styling, and/or coloring the hair.

In at least one embodiment, the composition may be a product for use in the field of haircare, for instance, products for holding the hair style, products for shaping the hair, and products for cleansing the hair. The hair compositions may be chosen, for example, from shampoos, hair conditioners, styling gels, care gels, care lotions, care creams, conditioning agents, hairsetting lotions, blow-drying lotions, and fixing and/or styling compositions such as lacquers and sprays. The lotions may be packaged in various forms, for example, in vaporizers, pump-dispenser bottles, and aerosol containers to allow application of the composition in vaporized form or in the form of a mousse.

The composition may also be chosen from hair coloring products; permanent-waving compositions, relaxing compositions, bleaching compositions, and rinse-out compositions to be applied before and/or after dyeing, bleaching, permanent-waving, and/or relaxing the hair or alternatively between the two steps of a permanent-waving or hair relaxing operation.

The composition according to the invention may also be chosen from care compositions, for example, moisturizing compositions, for the skin, the lips, and/or the integuments, and skin cleansing composition, for example, makeup-removing products and bath or shower gels.

The composition may also be in the form of an uncolored care product for treating the skin, for example, a product for moisturizing the skin, smoothing out the skin, depigmenting the skin, nourishing the skin, protecting the skin from sunlight, and/or giving the skin a specific treatment. According to this embodiment, the composition may further comprise at least one care active agent chosen from depigmenting agents, emollients, moisturizers, anti-seborrhoeic agents, antiacne agents, hair restorers, keratolytic agents and/or desquamating agents, antiwrinkle agents, tensioning agents, anti-irritants, calmatives, vitamins, screening agents, odor absorbers, and mixtures thereof.

The composition may also be chosen from body hygiene compositions, for example, deodorants and antiperspirant products, and hair-removing compositions.

The composition may also be in the form of a makeup product, such as a colored makeup product, for facial skin, bodily skin, and/or for the hair, for instance, foundations, optionally having care properties, blushers, makeup rouges, eyeshadows, concealer products, eyeliners; lip makeup products, for instance lipstick, optionally having care properties, lip glosses, and lip pencils; makeup products for the integuments, for instance, the nails, the eyelashes, such as mascara cakes, the eyebrows, and the hair; and a temporary tattoo product for bodily skin.

Also disclosed herein is a cosmetic process for treating, for instance, for making up, caring for, cleansing and/or coloring, keratin materials, such as bodily skin, facial skin, the nails, the hair, bodily hair, and/or the eyelashes, comprising applying to the materials at least one cosmetic composition of the present disclosure.

The application may optionally be followed by rinsing with water. Thus, the process according to the present disclosure may allow for hold of the hair style and/or the cosmetic treatment, care, makeup, washing, and/or makeup removal, inter alia, of the skin, the hair, and/or of any other keratin material.

Further disclosed herein is a process for improving both the persistence of at least one effect provided after deposition by a cosmetic composition and the adhesion of the composition applied to the keratin materials, and for allowing rapid, total, and selective removal of the deposit, comprising adding to the composition an effective amount of at least one (co)polymer as defined herein.

The removal of the deposit may comprise rinsing a cleansing composition or removing a deposit of makeup (for example, lipstick, foundation, mascara, and eyeliner). This removal may be performed using a hydrogen interaction disrupter.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below. Unless otherwise indicated, the amounts are given as percentage by weight.

### EXAMPLES

### Example 1

### Polyurethane-Polyester Copolymer with Ionizable Amine Units

1/ Preparation of a Monomer (I) of Structure G₁-L₁-A-L₂-G₁ of Formula:

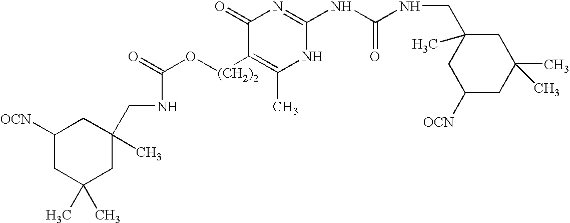

12 g of 5-(2-hydroxyethyl)-6-methylisocytosine were suspended in 150 ml of isophorone diisocyanate (IPDI) and the mixture was stirred under argon for 12 hours at 90° C.

The resulting clear solution was cooled and precipitated from hexane. The precipitate washed with hexane and then isolated by filtration, washing and drying. 46 g of solid were obtained, i.e. a yield of 93%.

2/ Preparation of a Polyurethane-Polyester Copolymer Bearing an Ionizable Function

16.3 g (1 eq.) of poly(2-methyl-1,3-propylene glutarate) with a number-average molecular weight Mn=1.0 kDa (OH-functionalized telechelic polymer) in 250 ml of chloroform were mixed with 7.1 g (1 eq.) of isophorone, 8.4 g (6/7 eq.) of monomer (1) prepared in the preceding step, and 3.6 g (1 eq.) of N-methyldiethanolamine (ionizable unit).

A few drops of dibutyltin dilaurate (DBTDL) were added and the mixture was then heated at 60° C. for 16 hours.

The polymer was precipitated from hexane and then dried under reduced pressure. The desired polymer was obtained in a yield of 96%. The number-average mass (Mn) of the polymer obtained was about 13 000 Da.

### Example 2

### Nail Varnish

4.8 g of the polymer prepared in Example 1 were dissolved in 30 ml of THF; 22 g of butyl acetate were added and the THF was then evaporated off.

A viscous solution containing 17.9% by weight of polymer was thus obtained. The viscosity may be reduced, if necessary, by adding 2 g of ethanol.

After applying this solution to the nails, a glossy transparent film was obtained.

### Example 3

### Hair Composition

16 g of the polymer prepared in Example 1 were dissolved in 64 g of THF: the medium was viscous.

10 g of ethanol and then 14.4 g of 1N HCl and 76.3 g of water were added, with stirring using a magnetic bar, at room temperature. The THF and the ethanol were evaporated off at 50° C. on a rotary evaporator.

A slightly yellowish transparent solution of low viscosity was obtained.

Final dry extract: 18.4% (by weight)

Particle size measured by light scattering (using a Coulter N4-SD machine) (after high dilution): 30 nm

pH=2.3

The solution was then be placed in a pump-dispenser bottle and vaporized on the hair.

Fixing of the hair was observed, which held over time; and the hair was shiny.

### Example 4

### (Meth)Acrylate Polymer with Ionizable Amine Units

1/ Preparation of a Monomer (I) of Structure G₁-L-A of Formula: (Known as “UPY Acrylate”)

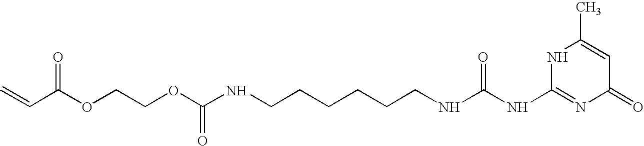

The monomer may be prepared by reacting hydroxyethyl acrylate with OCN—(CH₂)₆-ureidopyrimidone, as described in U.S. Patent Application Publication No. 2004-0034190 and may be represented schematically as follows:

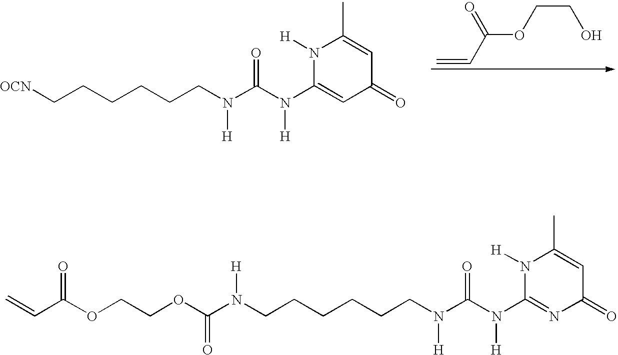

46 g of isocyanate were suspended in 1 litre of chloroform, and 36 ml of hydroxyethyl acrylate and 10 drops of dibutyltin dilaurate (DBTDL) were then added. The mixture was stirred at an oil bath temperature of 90° C. for 4 hours and was then cooled and filtered. The filtrate was concentrated and an excess of diethyl ether was added. The mixture was filtered and a white precipitate was obtained, which washed with diethyl ether and then dried under reduced pressure.

75 g of a solid white product were obtained, i.e. a yield of 91%.

2/ Preparation of a Copolymer Based on Dimethylaminoethyl Methacrylate/Butyl Acrylate/UPY Acrylate

The constituents below were reacted, in a mixture of 59.5 ml toluene/10.5 ml DMSO, at 60° C. under argon, for 8 hours:

| | | |
|---|---|---|
| Dimethylaminoethyl methacrylate | 10.5 | ml |
| Butyl acrylate | 24.5 | ml |
| UPY acrylate prepared above | 6.24 | g |
| AIBN (azoisobutyronitrile) initiator | 140 | mg |
| Transfer agent (dodecanethiol) | 1.4 | ml |

After reaction, the polymer solution was precipitated in water and the polymer was then recovered and dried in an oven for 12 hours.

3.94 g of polymer were obtained, i.e. a yield of 96%.

### Example 5

### Dispersion in Water/Hair Composition

10 g of the polymer prepared in Example 4 were dissolved in 50 g of THF: the medium was viscous.

16 ml of 1N HCl and 50 g of water were added, with stirring using a magnetic bar, at room temperature. The THF was evaporated off at 50° C. on a rotary evaporator.

A slightly yellowish transparent solution of low viscosity was obtained.

Final dry extract: 20% (by weight)

Particle size measured by light scattering (using a Coulter N4-SD machine) (after high dilution): 100 nm

pH=2.5

The solution was then be placed in a pump-dispenser bottle and vaporized on the hair.

Fixing of the hair was observed, which held over time; and the hair was shiny.

### Example 6

### Polyurethane-Polyester Copolymer Containing Ionizable Acid Units

20.7 g of dihydroxy telechelic polymer: poly(2-methyl-1,3-propylene adipate) of Mn=2.0 kD were dried for 2 hours under reduced pressure and then dissolved in 100 ml of a 1 vol/1 vol THF/methyl ethyl ketone mixture with 0.69 g of dimethylolpropionic acid (ionizable unit).

The mixture was heated to 80° C. under argon. Once a clear solution was obtained, 1.15 g of isophorone diisocyanate (IPDI) and 4 drops of dibutyltin dilaurate were added. The mixture was stirred and heated at 80° C., still under argon, for 2 hours.

5.46 g of monomer (I) prepared in Example 1 were then added and the mixture was stirred at 80° C. under argon for 16 hours. The reaction volume was reduced to half by evaporation on a rotavapor, diluted in 50 ml of toluene, and stirred under argon at 80° C. for a further 16 hours. Disappearance of the isocyanate band was confirmed by IR spectroscopy.

10 ml of methanol were then added and the desired polymer was precipitated from 1 litre of hexane. The product was dried under reduced pressure to give 26.8 g of polymer, i.e. a yield of 96%.

### Example 7

### Nail Varnish Application

8.2 g of the polymer prepared in Example 6 were dissolved in a mixture of 40 g of butyl acetate and 9 g of ethanol to obtain a viscosity adequate for a nail varnish application. After stirring at room temperature, a clear solution containing 13% by weight of polymer was obtained. After application to the nail, a glossy film was obtained.

### Example 8

### Polyurethane-PDMS Copolymer Containing Ionizable Amine Units

31 g of di-OH telechelic PDMS polymer, sold under the name KF003 by Shin-Etsu (Mn=5 kDa), were dissolved in 120 ml of dry toluene in the presence of 1.5 g of N-methyldiethanolamine (3.94% by weight of ionizable units), a catalytic amount of dibutyltin dilaurate and 2.8 g of isophorone diisocyanate. This solution was heated at 80° C. under argon for 2 hours.

30 ml of pyridine and 2.80 g of monomer (I) prepared in Example 1 were added thereto and the mixture was heated under argon for a further 16 hours.

The reaction medium was concentrated to 50% of its initial volume and then stripped successively with 2×50 ml of toluene. The reaction medium was then dissolved in a mixture of chloroform/methanol solvent (9 volumes/1 volume) and precipitated from methanol (methanol in ten fold excess by volume relative to the volume of the reaction medium).

38.5 g of final product were obtained after filtering and drying under reduced pressure.

This product was characterized by GPC (THF) Mn=14 kDa with a PD of 1.7.

### Example 9

### Makeup Application (Foundation and Lipstick)

The polymer of Example 8 was dissolved to a proportion of 20% in decamethylcyclopentasiloxane (D5).

After application to the skin, a comfortable film was obtained.

### Example 10

### Makeup Application (Foundation and Lipstick)

The polymer of Example 8 may also be partially protonated, in order to reduce the viscosity of a silicone oil solution comprising it.

Thus, a solution containing 10% polymer of Example 8 in decamethylcyclopentasiloxane (D5) was prepared. A solution of HCl (1M) in ether was added thereto (0.32 ml) per 1 g of polymer) in order to partially protonate the amine present on the polymer backbone. The solution was stirred for 3 hours at room temperature, with the flask open. The viscosity of the solution disappeared.

This oily solution may be applied to the skin. It formed a comfortable film.

What is claimed is:

1. A cosmetic and/or pharmaceutical composition comprising, in a physiologically acceptable medium, at least one (co)polymer comprising:
(a) a polymer backbone -POL- comprising at least two repeating units,
(b) at least one junction group (A) bonded to the polymer backbone and capable of establishing H bonds with at least one partner junction group, of identical or different chemical nature, each pairing of a junction group involving at least 3 H bonds,
wherein the at least one (co)polymer comprises at least one ionizable group.

2. The composition of claim 1, wherein each pairing of a junction group involves 4 H bonds.

3. The composition of claim 1, wherein the at least one (co)polymer results from the polymerization of at least one monomer of formula (I) and optionally of at least one monomer of formula (II) according to the reaction scheme: 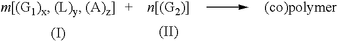
wherein:
the groups G₁, which may be identical or different, are (co)polymerizable groups capable of forming a covalent bond with another (co)polymerizable group G₁ of another monomer (I) and/or with a (co)polymerizable group borne by a monomer G₂;
the groups A, which may be identical or different, are junction groups capable of forming at least three H bonds;
the arms L, which may be identical or different, are divalent linker arms, including a single covalent bond, linking a junction group A to a group G₁;
x is an integer greater than or equal to 1;
y is an integer greater than or equal to 1;
z is an integer greater than or equal to 1;
G₂, which may be identical or different, are monomers free of a junction group A and comprising at least one (co)polymerizable group capable of forming a covalent bond with a (co)polymerizable group G₁ of a monomer (I) and/or with a (co)polymerizable group borne by another identical or different monomer G₂;
m is the number of moles of identical or different, homopolymerized or copolymerized monomers of formula (I) and is an integer ranging from 1 to 12;
n is the number of moles of identical or different, homopolymerized or copolymerized monomers of formula (II), and is an integer ranging from 0 to 20 000; and
m+n≧2;
wherein at least one of the groups A and/or groups G₁ and/or arms L and/or monomer G₂ comprises at least one ionizable group.

4. The composition of claim 3, wherein the groups A, which may be identical or different, are junction groups capable of forming 4 H bonds.

5. The composition of claim 3, wherein, in the at least one monomer of formula (I),
x is equal to 1 or 2,
y is equal to 1 or 2, and
z is equal to 1.

6. The composition of claim 3, wherein, in the at least one monomer of formula (I), m is an integer ranging from 2 to 6.

7. The composition of claim 3, wherein, in the at least one monomer of formula (II), n is an integer ranging from 1 to 5000.

8. The composition of claim 3, wherein the at least one (co)polymer is formed by reacting at least one monomer of formula G₁-L-A and/or G₁-L-A-L-G₁ and/or G₁-L(A)-G₁ with at least one monomer of formula G₂, wherein the groups G₁, the linker arms L, the junction groups A, and the monomers G₂ may be identical or different.

9. The composition of claim 1, wherein the at least one (co)polymer comprises at least 2 junction groups.

10. The composition of claim 9, wherein the at least one (co)polymer comprises at least 6 junction groups.

11. The composition of claim 1, wherein the at least one junction group (A) is a chemical group comprising at least 3 identical or different heteroatoms chosen from O, N, S, P, and F.

12. The composition of claim 11, wherein said at least one junction group (A) is a carbon-based group.

13. The composition of claim 12, wherein said at least one junction group (A) comprises four identical or different heteroatoms.

14. The composition of claim 1, wherein the at least one junction group (A) comprises at least 3 functional groups chosen from:

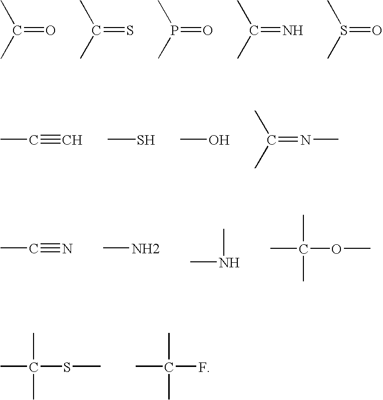

15. The composition of claim 1, wherein the at least one junction group (A) comprises 5- or 6-atom aromatic or unsaturated heterocyclic rings comprising C and/or N atoms, with conjugated double bonds.

16. The composition of claim 1, wherein the at least one junction group (A) is chosen from the following families and the tautomeric forms thereof:
(i) aminopyrimidones of formula: 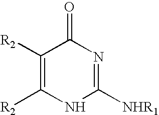
(ii) ureidopyrimidones of formula: 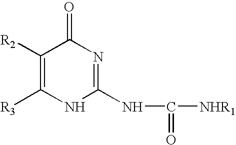
(iii) acylaminopyridines chosen from:
monoacylaminopyridines of structure: 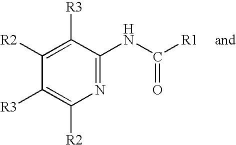
di(acylamino)pyridines of structure: 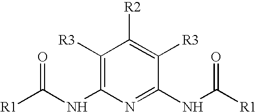
(iv) aminopyrimidines chosen from:
aminopyrimidine compounds: 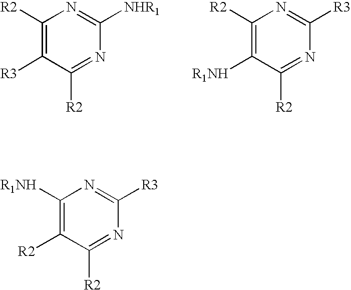
diaminopyrimidine compounds of structures: 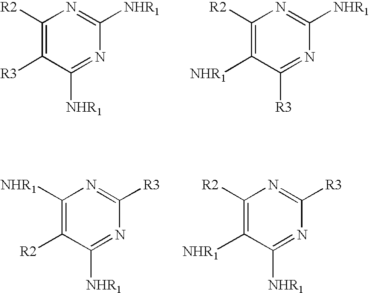
triaminopyrimidine compounds; and
ureidotriazines and ureidoaminotriazines of structure: 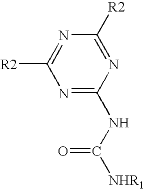
(vi) (acylamino)triazines chosen from:
di(acylamino)triazines of structure: 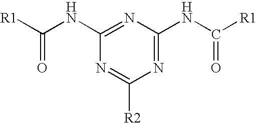
acylamino, aminotriazines, (mono- or diacylamino, and mono- or diamino) and compounds of structure: 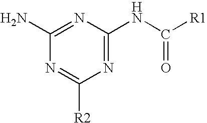
acylaminotriazines of structure: 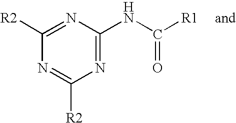
triaclaminotriazines,
(vii) aminotriazines chosen from:
monoaminotriazines,
2,6-diamino-s-triazines of structure: 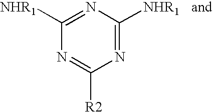
triamino-s-triazine compounds of structure: 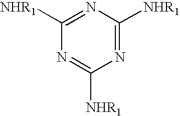
(viii) acylaminotriazoles of structure: 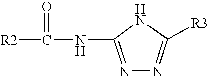
(ix) compounds of the urazoylbenzoic acid family of structure: 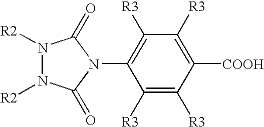
(x) phthalhydrazides of structure: 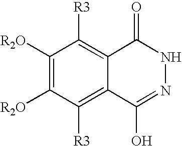
(xi) uracils of structure: 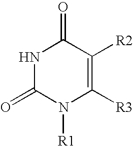
(xii) thymines of structure: 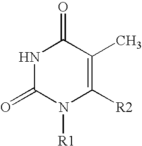
(xiii) succinimides of structure: 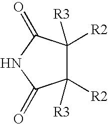
(xiv) glutarimides of structure: 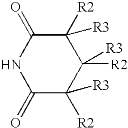
(xv) compounds of the cyanuric acid family of structure: 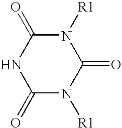
(xvi) maleimides: 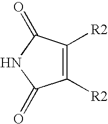
(xvii) compounds of the barbituric acid family of structure: 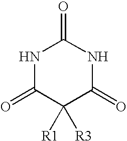
(xviii) compounds of structures: 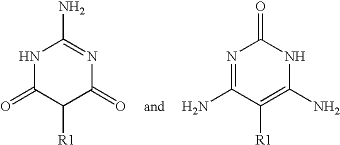
(xix) compounds of the trimellitic family, of formula: 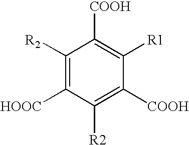
(xx) ureidopyridines of formulas: 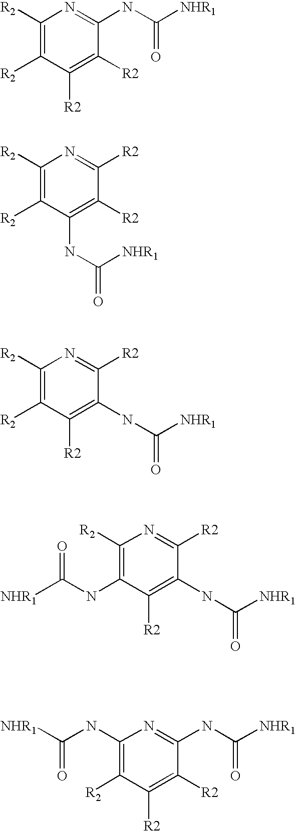
(xxi) carbamoylpyridines of formulas: 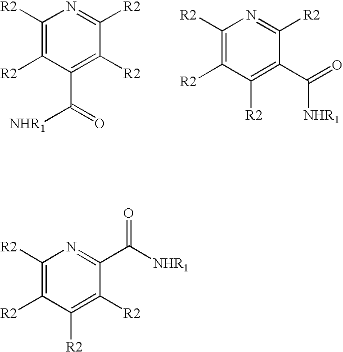
(xxii) adenines of formula: 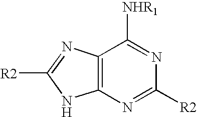
(xxiii) guanines of formula: 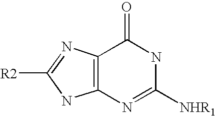
(xxiv) cytidines of formula: 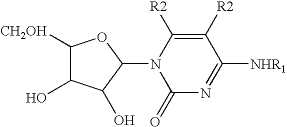
wherein in groups (i)-(xxiv):
(a) the radicals R¹, which may be identical or different, are chosen from H, halogen, and monovalent linear, branched, or cyclic, saturated or unsaturated, optionally aromatic C₁-C₆₀₀₀ carbon-based groups, which may optionally comprise at least one heteroatom chosen from O, S, N, P, Cl, Br, and F; or a combination of these meanings;
(b) the radicals R², which may be identical or different, are chosen from H, halogen, —OH, —N(R)₂, wherein R is chosen from H and linear or branched C₁-C₁₂ alkyl radicals; and monovalent linear, branched, or cyclic, saturated or unsaturated, optionally aromatic C₁-C₆₀₀₀ hydrocarbon-based groups, which may optionally comprise at least one heteroatom chosen from O, S, N, P, and F; or a combination of these meanings;
(c) the radicals R³, which may be identical or different, are chosen from H and monovalent linear, branched, or cyclic, saturated or unsaturated, optionally aromatic C₁-C₆₀₀₀ hydrocarbon-based groups, which may optionally comprise at least one heteroatom chosen from O, S, N, P, and F; or a combination of these meanings;
with the proviso that at least one of the groups R¹ and R² is the point of attachment of the junction group A to the polymer backbone -POL-.

17. The composition of claim 16, wherein R¹ is chosen from C₄-C₁₂ cycloalkyl groups, linear or branched C₁-C₃₀ alkyl group, and C₄-C₁₂ aryl groups, which are optionally substituted with at least one function chosen from amino, ester, and hydroxyl functions.

18. The composition of claim 16, in which:
i) R¹ is chosen from C₄H₉, phenyl, 1,4-nitrophenyl, 1,2-ethylene, 1,6-hexylene, 1,4-butylene, 1,6-(2,4,4-trimethyl-hexylene), 1,4-(4-methylpentylene), 1,5-(5-methyl-hexylene), 1,6-(6-methylheptylene), 1,5-(2,2,5-trimethylhexylene), 1,7-(3,7-dimethyloctylene); -isophorone-, 4,4′-methylenebiscyclohexylene, 2-methyl-1,3-phenylene, 4-methyl-1,3-phenylene, and 4,4-biphenylenemethylene groups; and/or
ii) R² is chosen from
H, CN, and NH₂,:
C₁-C₃₀ alkyl groups,
C₄-C₁₂ cycloalkyl groups,
C₄-C₁₂ aryl groups,
(C₄-C₁₂)aryl(C₁-C₃₀)alkyl groups,
C₁-C₄ alkoxy groups,
arylalkoxy groups,
C₄-C₁₂ heterocycles,
thioalkoxy groups,
sulfoxy groups, and
mixtures thereof;
wherein these groups may be optionally substituted with at least one function chosen from amino, ester, and hydroxyl functions; and/or
iii) R³ is chosen from C₄-C₁₂ cycloalkyl groups; linear or branched C₁-C₃₀ alkyl groups, and C₄-C₁₂ aryl groups; optionally substituted with at least one function chosen from amino, ester, and hydroxyl functions; and/or
iv) the point of attachment is borne by R₁ and/or R₂.

19. The composition of claim 16, wherein the at least one junction group (A) is chosen from:
(a) self-complementary and identical junction groups (A) chosen from:
aminopyrimidones and ureidopyrimidones,
compounds of the trimellitic acid family and of urazoylbenzoic acid,
acylaminopyridines, ureidopyridines, and carbamoylpyridines,
acylaminotriazines, ureidotriazines, and diaminotriazines,
acylaminotriazoles,
phthalhydrazides, and
compounds of formulas: 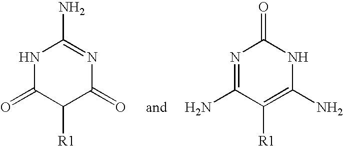
wherein R¹ is chosen from H and monovalent linear, branched, or cyclic, saturated or unsaturated, optionally aromatic, C₁-C₆₀₀₀ hydrocarbon-based groups, which may optionally comprise at least one heteroatom chosen from O, S, N, P, and F.
(b) complementary but different junction groups (A) chosen from:
adenine, which is complementary to guanine,
cytidine, which is complementary to thymine,
triamino-s-triazine, which is complementary to uracil, succinimide, glutarimide, cyanuric acid, thymine, maleimide, (di)aminopyrimidine, and barbituric acid; and
acylaminoamino-s-triazine, which is complementary to uracil, succinimide, glutarimide, cyanuric acid, thymine, maleimide, (di)aminopyrimidine, and barbituric acid.

20. The composition of claim 16, wherein the at least one junction group (A) is chosen from groups capable of establishing at least three H bonds with each other (self-complementary), chosen from:
ureidopyrimidones;
ureidopyridines and carbamoylpyridines;
acylamino-s-triazines;
ureidotriazines;
phthalhydrazines; and
compounds of formulas:

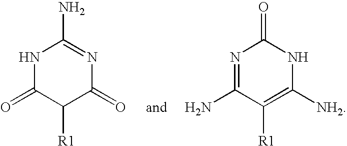

21. The composition of claim 16, wherein the at least one junction group (A) is chosen from:
2-ureidopyrimidone;
6-methyl, 2-ureidopyrimidone;
diacyl-2,6-diamino-s-triazine;
ureido-s-triazine; and
compounds of formulas: 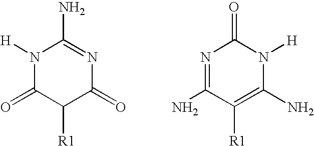
wherein R¹ is chosen from H and monovalent, linear, branched, or cyclic, saturated or unsaturated, optionally aromatic C₁-C₆₀₀₀ hydrocarbon-based groups which may optionally comprise at least one heteroatom chosen from O, S, N, P, and F.

22. The composition of claim 1, wherein the at least one junction group (A) bears at least one ionizable group.

23. The composition of claim 3, wherein the linker arm L is chosen from single covalent bonds and saturated or unsaturated, linear, branched, or cyclic, and aromatic divalent carbon-based groups, and combinations thereof, comprising from 1 to 6000 carbon atoms, and optionally comprising at least one identical or different heteroatom chosen from O, N, S, P, and F.

24. The composition of claim 23, wherein the divalent carbon based groups comprise from 1 to 30 carbon atoms.

25. The composition of claim 23, wherein the linker arms L are carbon-based groups optionally comprising at least one functional group chosen from:

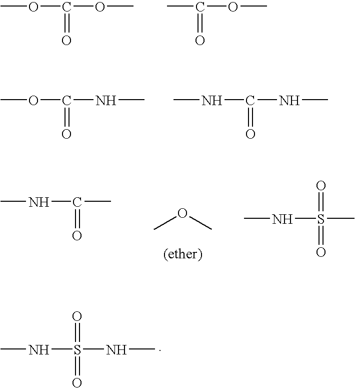

26. The composition of claim 23, wherein the linker arm L, when it is not a single bond, bears at least one ionizable group.

27. The composition of claim 3, wherein the (co)polymerizable group G₁ is chosen from:
(co)polymerizable groups comprising at least one ethylenic double bond capable of undergoing radical, anionic, or cationic (co)polymerization;
groups that may be (co)polymerized via nucleophilic or electrophilic substitution or addition, or radical addition, chosen from groups comprising at least one function chosen from hydroxyl (OH), activated hydroxyl, thiol (SH), halide (Br and Cl), primary and secondary amine (NH₂ and NHR), ester (COOR), carboxylic acid (COOH), activated acid, protected or unprotected isocyanate (NCO), isothiocyanate (NCS), —C═C—, —C(O)H, —SiH, succinimide, oxazoline, acetal, hemiacetal, chlorotriazine, —SO₂Cl, and epoxide functions; wherein the radical R is chosen from C₁-C₆ alkyls; and
groups that may be (co)polymerized via anionic or cationic ring opening, chosen from groups comprising at least one group chosen from cyclic ether, cyclic ester, cyclic amide, and cyclic carbonate groups.

28. The composition of claim 3, wherein the group G₁ bears at least one ionizable group.

29. The composition of claim 3, wherein the at least one monomer of formula (I) is chosen from:
i) monomers that may undergo radical, anionic, or cationic (co)polymerization, represented by the formula: 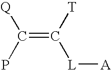
wherein P, Q, and T, which may be identical or different, are chosen from hydrogen, linear or branched, cyclic or non-cyclic, saturated or unsaturated, or aromatic, hydrocarbon-based radicals comprising from 1 to 12 carbon atoms, and a group -L-A;
ii) monomers that may be (co)polymerized via addition or polycondensation, comprising at least one group G₁, which can (co)polymerize via addition or polycondensation;
iii) monomers that (co)polymerize via ring opening, and comprising groups G₁ chosen from:
cyclic ethers of formula: 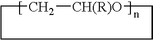
wherein R is chosen from H and linear or branched, cyclic or non-cyclic, saturated or unsaturated, or aromatic, hydrocarbon-based radicals, and n is an integer ranging from 1 to 3;
cyclic amides of formula: 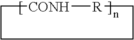
wherein R is —(CH₂)ₘ— and m is an integer ranging from 3 to 12, and n is an integer ranging from 1 to 3;
cyclic esters of formula: 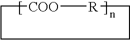
wherein R is —(CH₂)ₘ— and m is an integer ranging from 3 to 12, and n is an integer ranging from 1 to 3;
cyclic carbonates of formula: 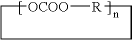
wherein R is —(CH₂)ₘ— and m is an integer ranging from 3 to 12, and n is an integer ranging from 1 to 3;
cyclic perfluoroethers, lactides, oxazolines, and norbornene, and derivatives thereof; and
combinations thereof.

30. The composition of claim 29, wherein the monomers that may be (co)polymerized via addition or polycondensation comprise one group chosen from A-L- and -L-A-L-.

31. The composition of claim 29, wherein the at least one monomer (I) is chosen from:
monomers that may undergo radical, anionic, or cationic (co)polymerization, of formula: 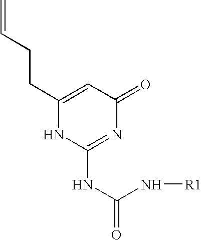
the acrylate derived from 6-methyl-2-ureidopyrimidone of formula: 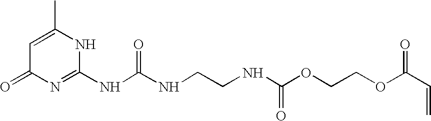
monomers of formulas: 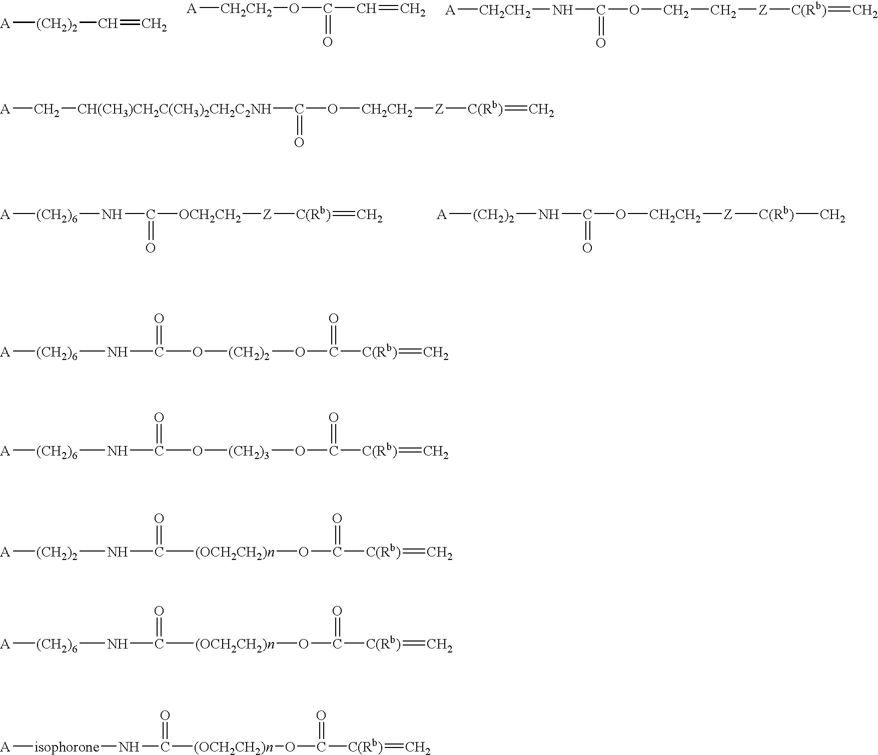
wherein
Z is chosen from —O—C(O)— and —NH—C(O)—;
n is an integer ranging from 1 to 500;
R^{b} is chosen from H and CH₃; and
A is the group: 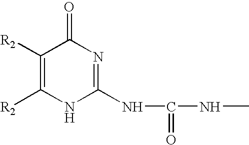
wherein R², which may be identical or different, is chosen from H, halogen, —OH, —N(R)₂, wherein R is chosen from H and linear or branched C₁-C₁₂ alkyl radicals; and monovalent linear, branched, or cyclic, saturated or unsaturated, optionally aromatic C₁-C₆₀₀₀ hydrocarbon-based groups, which may optionally comprise at least one heteroatom chosen from O, S, N, P, and F; or a combination of these meanings;
monomers that may be (co)polymerized via polyaddition or polycondensation, of formulas:

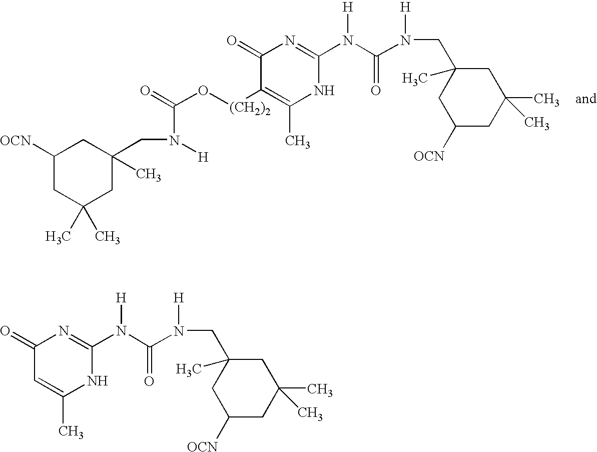

32. The composition of claim 3, wherein the at least one monomer (II) G₂ is an ethylenic monomer chosen from:
i) (meth)acrylates of formulas CH₂═CHCOOR⁴ and CH₂═C(CH₃)COOR⁴ in which R⁴ is chosen from:
hydrogen,
linear, cyclic, or branched alkyl groups comprising from 1 to 30 carbon atoms, in which is optionally intercalated at least one heteroatom chosen from O, N, S, and P, the alkyl groups also optionally being substituted with at least one substituent chosen from hydroxyl groups, halogen atoms, and groups Si(R₇)(R₈) wherein R₇ and R₈, which may be identical or different, are chosen from C₁-C₆ alkyl groups and phenyl groups;
C₃ to C₂₀ aryl groups;
C₄ to C₃₀ aralkyl and alkylaryl groups, wherein the alkyl group is C₁ to C₈;
C₄-C₁₂ heterocycloalkyl groups comprising at least one heteroatom chosen from O, N, P, and S, the ring being aromatic or non-aromatic;
C₄-C₃₀ alkylheterocycloalkyl groups, wherein the alkyl group is C₁ to C₈,
wherein the aryl and aralkyl groups may optionally comprise at least one intercalated heteroatom chosen from O, N, S, and P, and/or may be substituted with at least one substituent chosen from hydroxyl groups, halogen atoms, and linear or branched C₁-C₄ alkyl groups, which may themselves comprise at least one intercalated heteroatom chosen from O, N, S, and P and/or which may be substituted with at least one substituent chosen from hydroxyl groups, halogen atoms and groups Si(R₇)(R₈), wherein R₇ and R₈, which may be identical or different, are chosen from C₁ to C₆ alkyl groups and phenyl groups;
ii) (meth)acrylamides of formulas CH₂═CHCONR⁶R⁵ and CH₂—C(CH₃)CONR⁶R⁵ wherein R⁵ and R⁶, which may be identical or different, have the same meanings as for the groups R⁴ above;
iii) vinyl monomers of formulas: CH₂═CH—R⁹, CH₂═CH—CH₂—R⁹, and CH₂═C(CH₃)—CH₂—R⁹ wherein R⁹ is chosen from hydroxyl, halogen (Cl and F), NH₂, acetamide (—NHCOCH₃), —OR₁₀ (vinyl ether) groups wherein R₁₀ is chosen from phenyl groups and C₁-C₁₂ alkyl groups; —OCOR₁₁, (vinyl ester) groups wherein R₁₁ is chosen from:
(i) linear or branched C₂ to C₁₂ alkyl groups,
(ii) C₃ to C₁₂ cycloalkyl groups,
(iii) C₃ to C₂₀ aryl groups,
(iv) C₄ to C₃₀ aralkyl groups, wherein the alkyl group is C₁ to C₈,
(v) saturated or unsaturated, aromatic or non-aromatic, 4- to 12-membered heterocycloalkyl groups comprising at least one heteroatom chosen from O, N, and S, and
(vi) C₁ to C₄ alkylheterocycloalkyl groups,
wherein the alkyl, cycloalkyl, aryl, aralkyl, heterocycloalkyl, and alkylheterocycloalkyl groups are optionally substituted with at least one substituent chosen from hydroxyl groups, halogen atoms, and linear or branched C₁ to C₄ alkyl groups in which is optionally intercalated at least one heteroatom chosen from O, N, S, and P, the alkyl groups also optionally being substituted with at least one substituent chosen from hydroxyl groups, halogen atoms and groups Si(R₇)(R₈) wherein R₇ and R₈, which may be identical or different, are chosen from C₁ to C₆ alkyl groups and phenyl groups; and
iv) (meth)acrylate, (meth)acrylamide, and vinyl monomers comprising at least one group chosen from fluoro and perfluoro groups.

33. The composition of claim 32, wherein R⁴ is chosen from methyl, ethyl, propyl, n-butyl, isobutyl, tert-butyl, hexyl, ethylhexyl, octyl, lauryl, stearyl, isooctyl, isodecyl, and dodecyl groups; alkyl-based groups chosen from C₁-C₄ hydroxyalkyl groups; (C₁₋₄)alkoxy(C₁₋₄)alkyl groups; C₃ to C₁₂ cycloalkyl groups; t-butylbenzyl, phenyl, furfurylmethyl, tetrahydrofurfurylmethyl, and 2-ethylperfluorohexyl groups; the groups —(OC₂H₄)_{q}—OR, wherein q=5 to 500 and R is chosen from H and C₁ to C₃₀ alkyl radicals.

34. The composition of claim 32, wherein the at least one monomer (II) is chosen from (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide and N-(2-hydroxypropylmethacrylamide); vinylcyclohexane, styrene, N-vinylpyrrolidone, N-vinylcaprolactam; vinyl acetate, vinyl propionate, vinyl butyrate, vinyl ethylhexanoate, vinyl neononanoate, vinyl neododecanoate; methyl vinyl ether, ethyl vinyl ether, and isobutyl vinyl ether.

35. The composition of claim 32, wherein the at least one monomer (II) G₂ bears at least one ionizable group.

36. The composition of claim 3, wherein in the at least one monomer (I):
the group G-L-A-L-G is of the type: 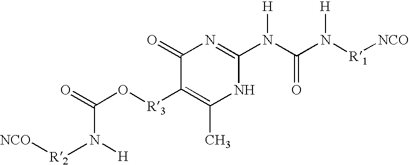
and/or
the group A-L-G is of the type: 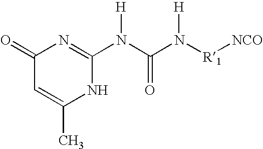
wherein the radicals R′₁, R′₂, and R′₃, which may be identical or different, are divalent carbon-based groups chosen from 1,2-ethylene, 1,6-hexylene, 1,4-butylene, 1,6-(2,4,4-trimethylhexylene), 1,4-(4-methylpentylene), 1,5-(5-methylhexylene), 1,6-(6-methylheptylene), 1,5-(2,2,5-trimethylhexylene), 1,7-(3,7-dimethyloctylene); -isophorone-, 4,4′-methylenebis(cyclohexylene), tolylene, 2-methyl-1,3-phenylene, 4-methyl-1,3-phenylene, and 4,4-bis(phenylene)methylene.

37. The composition of claim 36, wherein the radicals R′₁, R′₂, and R′₃, which may be identical or different, are chosen from isophorone, —(CH₂)₂—, —(CH₂)₆—, —CH₂CH(CH₃)—CH₂—C(CH₃)₂—CH₂—CH₂, 4,4′-methylenebis(cyclohexylene), and 2-methyl-1,3-phenylene.

38. The composition of claim 3, wherein the at least one ionizable group is borne by a linker arm L of the monomer (I) when L is not a single bond, or by the monomer (II) G₂ when it is present in the (co)polymer.

39. The composition of claim 3, wherein:
the at least one ionizable group is present in the (co)polymer in an amount ranging from 0.1% to 50% by weight relative to the total weight of the (co)polymer; and/or
the at least one monomer bearing the at least one ionizable group is present in the (co)polymer in an amount ranging from 3% to 20% by weight relative to the total weight of the (co)polymer.

40. The composition of claim 39, wherein the at least one ionizable group is present in the (co)polymer in an amount ranging from 1% to 15% by weight relative to the total weight of the (co)polymer.

41. The composition of claim 39, wherein the at least one monomer is present in the (co)polymer in an amount ranging from 8% to 15% by weight relative to the total weight of the (co)polymer.

42. The composition of claim 1, wherein the at least one ionizable group is chosen from:
i) anionizable groups, and salts thereof; and
ii) cationizable groups, and salts thereof.

43. The composition of claim 42, wherein said anionizable groups are chosen from groups comprising an acid function chosen from:
carboxylic radicals: —COOH,
sulfonic radicals: —SO₃H, —OSO₃H radicals,
phosphonic radicals: —(O)P(OH)₂,
phosphoric radicals: —OP(O)(OH)₂, and
organic or mineral salified forms thereof.

44. The composition according to claim 42, wherein said cationizable groups are chosen from groups comprising a function chosen from:
a) amine radicals of formula —N(R₁₅)(R₁₆) and organic or mineral salts thereof, wherein R₁₅ and R₁₆, which may be identical or different, are chosen from:
(i) hydrogen,
(ii) linear, branched, or cyclic, saturated or unsaturated, optionally aromatic alkyl groups comprising from 1 to 30 carbon atoms and optionally comprising from 1 to 10 heteroatoms chosen from O, N, S, and P;
(iii) alkylene oxide groups of formula —(R₁₇O)ᵣR₁₈ wherein R₁₇ is chosen from linear or branched C₂-C₄ alkyl radicals, R₁₈ is chosen from hydrogen and linear or branched C₁ to C₃₀ alkyl radicals, and r is a number ranging from 1 to 250;
wherein R₁₅ and R₁₆ may form, together with the nitrogen atom, a saturated or unsaturated, optionally aromatic ring comprising from 5 to 8 atoms chosen from carbon atoms and heteroatoms chosen from O, S, and N; the ring also optionally being fused with at least one other saturated or unsaturated, optionally aromatic ring comprising from 5 to 7 atoms chosen from carbon atoms and heteroatoms chosen from O, S, and N;
b) —R′₁₅—N—R′₁₆— groups wherein R′₁₅ and R′₁₆ form, together with the nitrogen atom, a saturated or unsaturated, optionally aromatic ring comprising 5 to 8 atoms chosen from carbon atoms and heteroatoms chosen from O, S, and N; the ring optionally being fused with at least one other saturated or unsaturated, optionally aromatic ring comprising from 5 to 7 atoms chosen from carbon atoms and heteroatoms chosen from O, S, and N;
c) guanidino groups and amidino groups;
d) quaternary ammonium radicals of formula: —N⁺(R²)₃Z⁻ wherein R¹², which may be identical or different, is chosen from linear or branched C₁ to C₂₀ alkyl radicals; and Z is chosen from halogen atoms and —OSO₃CH₃;
e) and mixtures thereof.

45. The composition of claim 1, wherein the at least one ionizable group is chosen from:
anionizable groups: monovalent groups —COOH, —CH₂COOH, —(CH₂)₂COOH, —(CH₂)₃COOH, —(CH₂)SO₃H, —(CH₂)₂SO₃H, —(CH₂)₃SO₃H, and —O(CH₂)₃SO₃H; and divalent groups —C(COOH)(CH₃)— and —CH₂—C(COOH)(CH₃)—CH₂—; and
cationizable groups: monovalent groups —(CH₂)₂—N(CH₃)₂, —N(CH₃)₂, (CH₂)₃—N(CH₃)₂, —O—(CH₂)₃—N(CH₂CH₃)₂, and —(CH₂)₂—N(CH₂CH₃)₂; and divalent groups —(CH₂)₂—N(CH₃)—(CH₂)₂— and —(CH₂)₃—N(CH₃)—(CH₂)₃—.

46. The composition of claim 3, wherein the group G₁ bears at least one ionizable group and is chosen from the groups CH₂═C(COOH)—COO— and HOC(CH₃)(COOH)—.

47. The composition of claim 46, wherein the group G₁-L-A is chosen from:
the group CH₂═C(COOH)—COO—CH₂OCONH(CH₂)₆— ureidopyrimidone of formula: 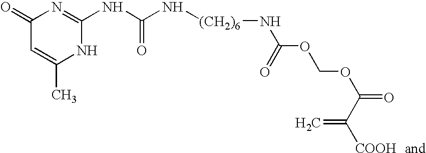
the group HOC(CH₃)(COOH)—CH₂CH₂OC(O)—NH(CH₂)₆— ureidopyrimidone of formula:

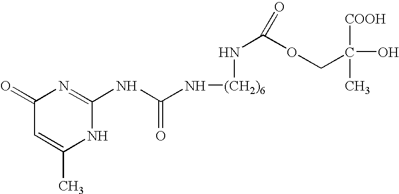

48. The composition of claim 3, wherein the at least one monomer (II) G₂ comprises at least one ionizable group and is chosen from:
(i) ethylenically unsaturated monomers comprising at least one function chosen from carboxylic acid (COOH), phosphonic acid (PO₃H₂), and sulfonic acid (SO₃H) functions,
(ii) ethylenically unsaturated monomers comprising at least one function chosen from primary, secondary, and tertiary amine functions,
(iii) monomers of formula: 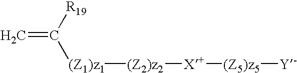
wherein:
R₁₉ is chosen from hydrogen and linear, cyclic, or branched hydrocarbon-based radicals of the type CₚH₂ₚ₊₁, wherein p is an integer ranging from 1 to 12;
Z₁ is a divalent group chosen from —COO—, —CONH—, —CONCH₃—, —OCO—, and —O—;
z₁ is equal to 0 or 1;
Z₂ is chosen from linear, branched, or cyclic, optionally aromatic, saturated or unsaturated divalent carbon-based radicals comprising from 1 to 30 carbon atoms, which may optionally comprise from 1 to 30 heteroatoms chosen from O, N, S, and P;
z₂ is equal to 0 or 1;
Z₅ has the same meaning as that given for Z₂, but may be different from Z₂,
z₅=0 or 1,
X′⁺ is a divalent group of formula —N⁺(R₆)(R₇)— wherein (i) R₆ and R₇, which may be identical or different, are chosen from hydrogen and linear, branched, or cyclic, optionally aromatic alkyl groups comprising from 1 to 25 carbon atoms, and optionally comprising from 1 to 20 heteroatoms chosen from O, N, S, and P; and (ii) R₆ and R₇ may form, together with the nitrogen atom, a first saturated or unsaturated, optionally aromatic ring comprising from 5 to 8 atoms chosen from carbon atoms and heteroatoms chosen from O, N and S; the first ring optionally being fused with at least one other saturated or unsaturated, optionally aromatic ring comprising from 5 to 8 atoms chosen from carbon atoms and heteroatoms chosen from O, N, and S; and
Y′⁻ is a group chosen from —COO⁻, —SO₃⁻, —OSO₃⁻, —PO₃²⁻, and —OPO₃²⁻;
(iv) monomers of formula HX″—B—X″H, wherein:
X″, which may be identical or different, is chosen from O, S, NH, and NR, wherein R is chosen from C₁₋₆ alkyl groups,
B is chosen from linear, branched, or cyclic, saturated or unsaturated, optionally aromatic alkylene radicals comprising from 1 to 6000 carbon atoms, optionally comprising at least one heteroatom chosen from O, S, P, and N, and/or optionally substituted with at least one atom chosen from fluorine and silicon atoms;
with the proviso that at least one of the radicals B bears at least one ionizable group.

49. The composition of claim 48, wherein said ethylenically unsaturated monomers comprising at least one function chosen from carboxylic acid (COOH), phosphonic acid (PO₃H₂), and sulfonic acid (SO₃H) functions is chosen from compounds of the following formula:
CH₂═C(R₁₉)-(Z₁)_{z1}-(Z₂)_{z2}-Y
wherein:
R₁₉ is chosen from hydrogen and linear, cyclic, or branched hydrocarbon-based radicals of the type CₚH₂ₚ₊₁, wherein p is an integer ranging from 1 to 12;
Z₁ is a divalent group chosen from —COO—, —CONH—, —CONCH₃—, —OCO—, and —O—;
z₁ is 0 or 1;
Z₂ is chosen from linear, branched, or cyclic, optionally aromatic, saturated or unsaturated divalent carbon-based radicals comprising from 1 to 30 carbon atoms, which may optionally comprise from 1 to 30 heteroatoms chosen from O, N, S, and P;
z₂ is 0 or 1; and
Y is a group chosen from —COOH, —SO₃H, —OSO₃H, —PO(OH)₂, and —OPO(OH)₂.

50. The composition of claim 48, wherein the ethylenically unsaturated monomers comprising at least one primary, secondary, or tertiary function are chosen from those of the following formula: 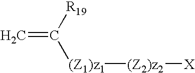
wherein:
R₁₉ is chosen from hydrogen and linear, cyclic, or branched hydrocarbon-based radicals of the type CₚH₂ₚ₊₁, wherein p is an integer ranging from 1 to 12;
Z₁ is a divalent group chosen from —COO—, —CONH—, —CONCH₃—, —OCO—, and —O—;
z₁ is 0 or 1;
Z₂ is chosen from linear, branched, or cyclic, optionally aromatic, saturated or unsaturated divalent carbon-based radicals comprising from 1 to 30 carbon atoms, which may optionally comprise from 1 to 30 heteroatoms chosen from O, N, S, and P;
z₂ is 0 or 1; and
X is a group of formula —N—R₁₇R₁₈ wherein R₁₇ and R₁₈, which may be identical or different, are chosen from,
(i) hydrogen;
(ii) linear, branched, or cyclic, saturated or unsaturated, optionally aromatic alkyl groups comprising from 1 to 30 carbon atoms, which may optionally comprise from 1 to 10 heteroatoms chosen from O, N, S, and P;
(iii) alkylene oxide groups of formula —(R₂₀O)_{y}R₂₁ wherein R₂₀ is chosen from linear or branched C₂-C₄ alkyl radicals, R₂₁ is chosen from hydrogen and linear or branched C₂-C₃₀ alkyl radicals, and y is a number ranging from 1 to 250;
(iv) R₁₇ and R₁₈ may form, together with the nitrogen atom, a saturated or unsaturated optionally aromatic ring comprising from 5 to 8 atoms chosen from carbon atoms and heteroatoms chosen from O, S, and N; the ring also optionally being fused with at least one other saturated or unsaturated, optionally aromatic ring comprising from 5 to 7 atoms chosen from carbon atoms and heteroatoms chosen from O, S, and N;
or alternatively X is a group —R′₁₅—N—R′₁₆— wherein R′₁₅ and R′₁₆ form, together with the nitrogen atom, a saturated or unsaturated, optionally aromatic ring, comprising from 5 to 8 atoms chosen from carbon atoms and heteroatoms chosen from O, S, and N; the ring optionally being fused with at least one other saturated or unsaturated, optionally aromatic ring comprising from 5 to 7 atoms chosen from carbon atoms and heteroatoms chosen from O, S, and N.

51. The composition of claim 48, wherein:
i) Z₂ is chosen from:
alkylene radicals chosen from methylene, ethylene, propylene, n-butylene, isobutylene, tert-butylene, n-hexylene, n-octylene, n-dodecylene, n-octadecylene, n-tetradecylene, and n-docosanylene;
phenylene radicals —C₆H₄-(ortho, meta, or para), which may be optionally substituted with a C₁-C₁₂ alkyl radical optionally comprising from 1 to 8 heteroatoms chosen from O, N, S, and P;
benzylene radicals —C₆H₄—CH₂—, which may be optionally substituted with a C₁-C₁₂ alkyl radical optionally comprising from 1 to 8 heteroatoms chosen from O, N, S, and P;
radicals of formulas —CH₂—CH(OH)—, —CH₂—CH₂—CH(OH)—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, and —CH₂—CH═CH— wherein R′ and R″, which may be identical or different, are chosen from C₁-C₁₈ alkyl radicals;
and/or
ii) X is chosen from aromatic or non-aromatic rings comprising a cationizable tertiary amine group and from aromatic or non-aromatic heterocycles comprising a cationizable tertiary nitrogen; and/or
iii) Z₂ and Z₅, which may be identical or different, are chosen from saturated or unsaturated linear, branched, or cyclic (aromatic or non-aromatic) alkylene groups comprising from 1 to 30 carbon atoms, and optionally including at least one heteroatom chosen from O, N, S, and P; and/or
iv) B is chosen from:
alkylene radicals comprising from 1 to 40 carbon atoms and cycloalkylene radicals comprising from 3 to 16 carbon atoms, optionally substituted with a C₁-C₁₂ alkyl radical and/or optionally comprising from 1 to 8 heteroatoms chosen from O, N, S, F, Si, and P;
C₁-C₃₀ arylene radicals;
C₁ to C₃₀ alkylarylene and arylalkylene radicals optionally substituted with a C₁-C₁₂ alkyl radical optionally comprising from 1 to 25 heteroatoms chosen from O, N, S, F, Si, and P;
and B may comprise at least one radical chosen from:
radicals of formulas —O—CO—O—, —CO—O—, —OCO—, —O—CO—NH—, anhydride, —NH—CO—NH—, and NHCO;
radicals —Si(R₄)(R₅)O— wherein R₄ and R₅, which may be identical or different, are chosen from H and linear or branched, cyclic or non-cyclic, saturated or unsaturated, or aromatic hydrocarbon-based radicals optionally comprising at least one identical or different heteroatom chosen from O, N, S, P, F, and Si; and/or
oxyalkylene and aminoalkylene radicals.

52. The composition of claim 48, wherein the at least one monomer (II) G₂ is chosen from:
a) acrylic acid, methacrylic acid, crotonic acid, itaconic acid, fumaric acid, maleic acid, diacrylic acid, dimethylfumaric acid, citraconic acid, acrylamidopropanesulfonic acid, 2-acrylamido-2-methylpropanesulfonic acid, styrenesulfonic acid, vinylbenzoic acid, vinylphosphoric acid, vinylsulfonic acid, vinylbenzenesulfonic acid, acrylamidoglycolic acid of formula CH₂═CH—CONHCH(OH)COOH, vinylphosphonic acid; 2-carboxyethyl (meth)acrylate, sulfopropyl methacrylate or acrylate (CH₂═C(CH₃)CO₂(CH₂)₃SO₃H), sulfoethyl(meth)acrylate, vinyl methyl sulfone, 2-(methacryloyloxy)ethyl phosphate of formula CH₂═C(CH₃)COOC₂H₄OP(O)(OH)₂; diallyl maleate of formula C₃H₅—CO₂—CH═CH—CO₂—C₂H₅; carboxylic anhydrides bearing a vinyl bond, and the salts thereof;
b) 2-vinylpyridine, 4-vinylpyridine, allylamine, and allylpyridine;
aminoalkyl(meth)acrylates;
aminoalkyl(meth)acrylamides;
vinylamine, vinylimidazole, and 2-(diethylamino)ethylstyrene;
N-vinylimidazole, N-vinyl-2-methylimidazole, and N-vinylcarbazole; and
the salts thereof and/or the quaternized forms thereof;
c) N,N-dimethyl-N-(2-methacryloyloxyethyl)-N-(3-sulfopropyl)ammonium betaine, N,N-dimethyl-N-(3-methacrylamidopropyl)-N-(3-sulfopropyl)ammonium betaine, and 1-(3-sulfopropyl)-2-vinylpyridinium betaine;
d) dimethylolpropionic acid, dimethylaminopropionic acid, N-ethylsulfonicdimethanolamine, N-ethylsulfonicdiethanolamine, benzenesulfonic acid diol, diaminopyridine, N-methyldiethanolamine, N-ethyldiethanolamine, and N-tert-butyldiethanolamine, and
e) mixtures thereof.

53. The composition of claim 1, wherein the at least one (co)polymer is chosen from those of the following structures:
linear (co)polymers α,ω-functionalized with junction groups (A);
linear (co)polymers comprising more than two junction groups, located in the chain and/or at one or both ends and/or as branches; and/or
branched (co)polymers with junction groups in the chain and/or as branches and/or at one or both ends.

54. The composition of claim 1, wherein the at least one (co)polymer is chosen, with the proviso that it comprises at least one ionizable group, from:
polyurethanes, polyureas, aliphatic or aromatic polyesters, aliphatic or aromatic polyamides, and copolymers thereof;
ethylenic (co)polymers chosen from (meth)acrylic copolymers, (meth)acrylamide copolymers, allylic copolymers, copolyolefins, and mixtures thereof;
(co)polymers obtained via ring opening;
polycaprolactams and polypyrrolidones;
siloxane (co)polymers;
polythioethers, polycarbonates, polyacetals, and perfluoropolyethers;
(co)polymers obtained via metathesis;
copolymers of these various types of polymers;
the salts thereof and derivatives thereof; and
mixtures thereof.

55. The composition of claim 54, wherein the (co)polymers obtained via ring opening are chosen from polyethylene oxide, polypropylene oxide and copolymers thereof (polyethylene oxide/polypropylene oxides); polylactides, polyesters, and polyoxazolines.

56. The composition of claim 1, wherein the at least one (co)polymer has a number-average molecular mass (Mn) ranging from 1000 to 3 000 000.

57. The composition of claim 56, wherein the at least one (co)polymer has a number-average molecular mass (Mn) ranging from 8000 to 500 000.

58. The composition of claim 1, wherein the at least one (co)polymer is soluble and/or dispersible in the medium of the composition.

59. The composition of claim 58, wherein the at least one (co)polymer is soluble in aqueous media, carbon-based oils, silicone oils, and/or cosmetic solvents.

60. The composition of claim 1, wherein the at least one (co)polymer is present in the composition in an amount ranging from 0.01% to 90% by weight of solids relative to the total weight of the composition.

61. The composition of claim 60, wherein the at least one (co)polymer is present in the composition in an amount ranging from 1% to 50% by weight of solids relative to the total weight of the composition.

62. The composition of claim 1, wherein the physiologically acceptable medium comprises a compound chosen from water, alcohols, polyols, esters, liquid ketones, propylene glycol ethers that are liquid at room temperature, ethers that are liquid at 25° C., alkanes that are liquid at 25° C., aldehydes that are liquid at 25° C., cyclic aromatic compounds that are liquid at 25° C., carbon-based oils, silicone oils, fluorosilicone oils, waxes, gums, surfactants, thickeners, hydrophilic or lipophilic gelling agents, hydrophilic or lipophilic cosmetic active agents, preserving agents, antioxidants, fragrances, nacreous agents, fillers, neutralizers, polymers other than the at least one (co)polymer, film-forming polymers, emulsifiers, co-emulsifiers, pigments, dyestuffs, nacres, and mixtures thereof.

63. The composition of claim 1, wherein the composition is in a form chosen from aqueous, alcoholic, or aqueous-alcoholic solutions, dispersions, and suspensions and oily solutions, which are optionally thickened or gelled; oil-in-water emulsions, water-in-oil emulsions, and multiple emulsions, of liquid or semi-liquid consistency of the milk type or of soft consistency of cream type; aqueous or anhydrous gels and mousses; oily or emulsified gels; dispersions of vesicles; two-phase or multiphase lotions; sprays; and any other cosmetic form.

64. The composition of claim 1, wherein the composition is in a form chosen from products for caring for, cleansing, and/or making up bodily and/or facial skin, the lips, and/or the hair, antisun and/or self-tanning products, body hygiene products, and hair products for caring for, cleansing, styling, and/or dyeing the hair.

65. The composition of claim 1, wherein the composition is chosen from:
hair compositions chosen from shampoos, hair conditioners, styling and/or care gels, care lotions and creams, conditioning agents, hairsetting lotions, blow-drying lotions, fixing and/or styling compositions; hair coloring products; compositions for permanent-waving, relaxing, and/or bleaching the hair; rinse-out compositions to be applied before and/or after dyeing, bleaching, permanent-waving, and/or relaxing the hair or alternatively between the two steps of a permanent-waving or hair-relaxing operation;
care compositions chosen from moisturizing compositions for the skin, the lips, and/or the integuments,
skin cleansing compositions chosen from makeup-removing products and bath or shower gels;
uncolored care products for treating the skin, moisturizing the skin, smoothing the skin out, depigmenting the skin, nourishing the skin, protecting the skin from sunlight, and/or giving the skin a specific treatment;
body hygiene compositions chosen from deodorants, antiperspirant products, and hair-removing compositions;
makeup products for bodily and/or facial skin and/or the hair chosen from foundations, optionally having care properties, blushers, makeup rouges, eyeshadows, concealer products, eyeliners; lip makeup products, optionally with care properties, lip gloss; lip pencils; makeup products for the integuments; and temporary tattoo products for bodily skin.

66. A cosmetic treatment process for making up, caring for, cleansing, and/or coloring keratin materials comprising applying to the materials at least one cosmetic and/or pharmaceutical composition comprising, in a physiologically acceptable medium, at least one (co)polymer comprising:
(a) a polymer backbone -POL- comprising at least two repeating units,
(b) at least one junction group (A) bonded to the polymer backbone and capable of establishing H bonds with at least one partner junction group, of identical or different chemical nature, each pairing of a junction group involving at least 3 H bonds,
wherein the at least one (co)polymer comprises at least one ionizable group.

67. A process for improving both the persistence of at least one effect provided after deposition by a cosmetic composition and the adhesion of the composition applied to keratin materials, and for allowing rapid, total, and selective removal of the deposit, comprising adding to the composition an effective amount of at least one (co)polymer comprising:
(a) a polymer backbone -POL- comprising at least two repeating units,
(b) at least one junction group (A) bonded to the polymer backbone and capable of establishing H bonds with at least one partner junction group, of identical or different chemical nature, each pairing of a junction group involving at least 3 H bonds,
wherein the at least one (co)polymer comprises at least one ionizable group.
